SEQUENCE LISTING (9 sequences)

SEQ ID NO: 1 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 2 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 3 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 4 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 6 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 7 - protein, 354 aa, Homo sapiens
MRLAVLFSGALLGLLAAQGTGNDCPHKKSATLLPSFTVTPTVTESTGTTSHRTTKSHKTTTHRTTTTGTTSHGPTTATHNPTTTSHGNVTVHPTSNSTATSQGPSTATHSPATTSHGNATVHPTSNSTATSPGFTSSAHPEPPPPSPSPSPTSKETIGDYTWTNGSQPCVHLQAQIQIRVMYTTQGGGEAWGISVLNPNKTKVQGSCEGAHPHLLLSFPYGHLSFGFMQDLQQKVVYLSYMAVEYNVSFPHAAQWTFSAQNASLRDLQAPLGQSFSCSNSSIILSPAVHLDLLSLRLQAAQLPHTGVFGQSFSCPSDRSILLPLIIGLILLGLLALVLIAFCIIRRRPSAYQAL

SEQ ID NO: 8 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 9 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2025/0093356 A1
(43) Pub. Date: Mar. 20, 2025

(54) METHODS AND SYSTEMS FOR PREDICTING RESPONSE TO PD-1 AXIS DIRECTED THERAPEUTICS IN COLORECTAL TUMORS WITH DEFICIENT MISMATCH REPAIR

(71) Applicants: Ventana Medical Systems, Inc., Tucson, AZ (US); Mayo Foundation for Medical Education and Research, Rochester, MN (US)

(72) Inventors: Eze O. AHANONU, Tucson, AZ (US); Sara Marie MCMAHON, Vail, AZ (US); Kandavel SHANMUGAM, Oro Valley, AZ (US); Frank A. SINICROPE, Rochester, MN (US); Crystal Sue WILLIAMS, Ithaca, NY (US); Dongyao YAN, Oro Valley, AZ (US); Wenjun ZHANG, Tucson, AZ (US)

(73) Assignees: Ventana Medical Systems, Inc., Tucson, AZ (US); Mayo Foundation for Medical Education and Research, Rochester, MN (US)

(21) Appl. No.: 18/899,219

(22) Filed: Sep. 27, 2024

Related U.S. Application Data
(63) Continuation of application No. PCT/US23/65155, filed on Mar. 30, 2023.
(60) Provisional application No. 63/362,305, filed on Mar. 31, 2022.
(60) Provisional application No. 63/383,688, filed on Nov. 14, 2022.

(51) Int. Cl. G01N 33/574; G06T 7/00; G06T 7/73; G06V 10/25; G06V 20/69; G06V 20/70

(52) U.S. Cl. CPC G01N 33/57419; G06T 7/0014; G06T 7/74; G06V 10/25; G06V 20/695; G06V 20/698; G06V 20/70; G01N 2333/4742; G01N 2333/70517; G01N 2333/70521; G01N 2333/70532; G01N 2333/70596; G01N 2474/20; G06T 2207/30024; G06T 2207/30096; G06T 2207/30204; G06V 2201/03

(57) ABSTRACT

Scoring functions for predicting response of a dMMR and/or MSI-H colorectal tumor to a PD-1 axis-directed therapy are disclosed, as well as methods and systems for evaluating tissue samples for the presence of feature metrics useful in computing such scoring functions. The scoring functions integrate one or more spatial relationships between cell types into a numerical indication of the likelihood that the tumor will respond to the PD-1 axis-directed therapy. Based on the output of the scoring function, a subject may then be selected to receive a PD-1 axis-directed therapy (if the scoring function indicates a sufficient likelihood of positive response) or an alternative therapy (if the scoring function indicates an insufficient likelihood of positive response).

### CROSS-REFERENCE TO RELATED APPLICATIONS

This application is a continuation of PCT Application No. PCT/US2023/065155, filed Mar. 30, 2023, which claims the benefit of priority of U.S. Provisional Application Nos. 63/362,305, filed Mar. 31, 2022, and 63/383,688, filed Nov. 14, 2022, both of which are incorporated by reference herein in their entirety for any purpose.

### SEQUENCE LISTING INCORPORATION-BY-REFERENCE

A Sequence Listing entitled “P37479WO_SEQ_LIST,” created on Feb. 1, 2023, and 10,677 bytes in size is hereby incorporated by reference.

### BACKGROUND

### Field of the Invention

The invention relates to detection, characterization and enumeration of biomarkers in tumor samples useful for predicting response to immune checkpoint inhibitor therapy.

### Brief Description of Related Art

Programmed death ligand 1 (PD-L1) is an immune checkpoint protein that regulates the immune system through binding of the programmed cell death protein 1 (PD-1) receptor. PD-L1 is expressed on multiple immune cell types and is also expressed in many cancer cell types, including colorectal cancer (CRC) cells. PD-L1 can bind to PD-1 receptors on activated T cells, which leads to the inhibition of the cytotoxic T cells and enables immune evasion of cancer. See Zou et al (2016). Cancers may escape immune surveillance and eradication through the up-regulation of the programmed death 1 (PD-1) pathway, and its ligand, programmed death-ligand 1 (PD-L1), on tumor cells and in the tumor microenvironment. Blockade of this pathway with antibodies to PD-1 or PD-L1 has led to remarkable clinical responses in some cancer patients. However, identification of predictive biomarkers for patient selection represents a major challenge.

CRCs with deficient DNA mismatch repair (dMMR) have microsatellite instability (MSI) that results in hypermutation and expression of mutation-specific neopeptides. See Llosa et al. (2015). Treatment of metastatic CRCs (mCRC) with the anti-PD-1 antibody, pembrolizumab, produced frequent and durable responses in these patients which led to its approval by the U.S. Food and Drug Administration for this tumor subgroup after progression following treatment with a fluoropyrimidine, oxaliplatin, and irinotecan. However, more than half of dMMR mCRC patients display resistance to PD-1 blockade due to mechanisms that remain unknown. See Le (I) & Le (II).

PD-L1 is the most widely used predictive biomarker for selection of patients to receive PD-1 axis directed therapeutics. However, tumor type-related differences are observed and to date, PD-L1 expression has not been useful for prediction of treatment response in patients with colorectal cancer. See Yi.

The evaluation of the intra-tumoral immune infiltrate has been proposed as a promising area of investigation for potential biomarker signatures relating to immunotherapies. The presence or absence as well as the intensity of an inflammatory response is known to be a prognostic factor in a number of different cancer types, including colorectal cancer. See Jass I; Jass II; Galon I; Galon II; Pagès. Additionally, spatial metrics between specific immune cell types have been investigated in colorectal tumors for their impact on prognosis, survival, and response to treatment. See Barrera; Chakrabarti, Wang I; Wang II; Yoon; Zhang I; WO 2020/072348 A1; WO 2020/161125 A1.

To date, however, no biomarker has yet been validated to accurately predict response to PD-1 blockade in patients with dMMR tumors. A need still remains to identify biomarker signatures that are indicative of response and benefit of immune checkpoint inhibitors in patients with dMMR CRCs.

### SUMMARY OF THE INVENTION

The present invention relates generally to scoring functions for predicting response of a dMMR and/or MSI-H colorectal tumor (including stage III and stage IV tumors) to a PD-1 axis-directed therapy, as well as methods and systems for evaluating tissue samples for the presence of feature metrics useful in computing such scoring functions. The scoring functions integrate one or more spatial relationships between cell types into a numerical indication of the likelihood that the tumor will respond to the PD-1 axis-directed therapy. Based on the output of the scoring function, a subject may then be selected to receive a PD-1 axis-directed therapy (if the scoring function indicates a sufficient likelihood of positive response) or an alternative therapy (if the scoring function indicates an insufficient likelihood of positive response).

In an exemplary embodiment, a method of treating a subject having a dMMR or MSI-H stage III colorectal tumor is provided, the method comprising administering to the subject a PD-1 axis-directed therapy, wherein the tumor has previously been determined to have an Predicted Response Score (PRS) indicative of response to the PD-1 axis-directed therapy and wherein the PRS is determined from a continuous scoring function incorporating a feature set selected from the group consisting of Feature Set 1, Feature Set 2, Feature Set 3, Feature Set 4, and Feature Set 5. In another embodiment, the continuous scoring function is a Cox proportional hazard model, such as a regularized Cox regression with LASSO.

In an embodiment, a method of treating a subject having a dMMR or MSI-H stage IV colorectal tumor, the method comprising administering to the subject a PD-1 axis-directed therapy, wherein the tumor has previously been determined to have an Predicted Response Score (PRS) indicative of response to the PD-1 axis-directed therapy and wherein the PRS determined from a continuous scoring function incorporating a feature set selected from the group consisting of Feature Set 1, Feature Set 2, Feature Set 3, Feature Set 4, and Feature Set 5. In another embodiment, the continuous scoring function is a Cox proportional hazard model, such as a regularized Cox regression with LASSO.

In an exemplary embodiment, the spatial relationship is a metric indicating the number of PD-1⁺ cells within a pre-defined distance of PD-L1⁺ cells (for example, average or median number of cells), which may be used standing alone or incorporated into a multivariate scoring function (such as a continuous scoring function) for prediction of response to the PD-1 axis directed therapeutic. In a specific embodiment, a method of treating a subject having a dMMR or MSI-H stage III or stage IV colorectal tumor is provided, the method comprising administering to the subject a PD-1 axis-directed therapy, wherein the tumor has previously been determined to have an average number of PD-1⁺ cells within a pre-defined distance (such as in the range of 5 μm to 50 μm) of a PD-L1⁺ cell that exceeds a pre-determined cutoff.

Also disclosed herein are systems, materials, and methods useful in making such predictions, including affinity histochemical assays and reagents, biomarker-specific reagent panels useful for performing such AHC assays, stained samples and slides, image analysis systems programmed to extract features from stained samples, etc.

Other features and embodiments will be apparent from the following detailed description.

### BRIEF DESCRIPTION OF THE DRAWINGS

FIG. 1A is a flow chart showing an exemplary method of selecting patients to receive a PD-1 axis directed therapy or an alternate therapy using the scoring functions disclosed herein.

FIG. 1B is a flow chart showing an exemplary method of selecting patients to receive a PD-1 axis directed therapy or an alternate therapy using the average number of PD-1⁺ cells within a pre-defined distance of at least 1 PD-L1 cell.

FIG. 2 illustrates exemplary components and modules of an image analysis system as disclosed herein.

FIG. 3A illustrates an exemplary workflow for performing object identification and ROI annotation tasks in an image analysis system as disclosed herein, wherein the object identification task is performed before (or simultaneously with) ROI annotation. A digital image of an AHC-stained tissue section is obtained (top panel) and the object identification task (such as that performed by the FI module as disclosed herein) is performed on substantially the whole image (middle panel). ROI annotation (bottom panel) does not affect the area of the image on which the object identification task is performed.

FIG. 3B illustrates an exemplary workflow for performing object identification and ROI annotation tasks in an image analysis system as disclosed herein, wherein the object identification task is performed after ROI annotation. A digital image of an AHC-stained tissue section is obtained (top panel) and the ROI Annotation task is performed (middle panel). The image identification task is only performed on the annotated (bottom panel) does not affect the area of the image on which the object identification task is performed.

FIG. 4 illustrates examples of additional systems that may be included with the image analysis systems to form a sample analysis system. Dashed arrows indicate sample flow. Solid arrows indicate data flow, including images of samples, process instructions, data outputs (such as feature metrics and PRS).

FIG. 5A illustrates the Predicted Response Score (PRS) distribution of a Cox proportional hazard model trained on Feature Set 1. The black vertical line illustrates the median PRS. The white bars and solid curve illustrate individuals falling in the low risk group (i.e. responders). The gray bars and hatched curve illustrate individuals falling in the high risk group (i.e. non-responders).

FIG. 5B illustrates a Kaplan-Meier curve stratified on the basis of the PRS risk group (low risk in light gray; high risk in dark gray), with the associated patient stratification chart at the bottom. “Time” on the X-axis illustrates days of overall survival after treatment.

FIG. 6A illustrates the Predicted Response Score (PRS) distribution of a Cox proportional hazard model trained on Feature Set 2. The black vertical line illustrates the median PRS. The white bars and solid curve illustrate individuals falling in the low risk group (i.e. responders). The gray bars and hatched curve illustrate individuals falling in the high risk group (i.e. non-responders).

FIG. 6B illustrates a Kaplan-Meier curve stratified on the basis of the PRS risk group (low risk in light gray; high risk in dark gray), with the associated patient stratification chart at the bottom. “Time” on the X-axis illustrates days of overall survival after treatment.

FIG. 7A illustrates the Predicted Response Score (PRS) distribution of a Cox proportional hazard model trained on Feature Set 3. The black vertical line illustrates the median PRS. The white bars and solid curve illustrate individuals falling in the low risk group (i.e. responders). The gray bars and hatched curve illustrate individuals falling in the high risk group (i.e. non-responders).

FIG. 7B illustrates a Kaplan-Meier curve stratified on the basis of the PRS risk group (low risk in light gray; high risk in dark gray), with the associated patient stratification chart at the bottom. “Time” on the X-axis illustrates days of progression-free survival after treatment.

FIG. 8A illustrates the Predicted Response Score (PRS) distribution of a Cox proportional hazard model trained on Feature Set 4. The black vertical line illustrates the median PRS. The white bars and solid curve illustrate individuals falling in the low risk group (i.e. responders). The gray bars and hatched curve illustrate individuals falling in the high risk group (i.e. non-responders).

FIG. 8B illustrates a Kaplan-Meier curve stratified on the basis of the PRS risk group (low risk in light gray; high risk in dark gray), with the associated patient stratification chart at the bottom. “Time” on the X-axis illustrates days of progression-free survival after treatment.

FIG. 9 illustrates a Kaplan-Meier curve stratified on the basis of the average number of PD-1 cells within 10 μm of at least 1 PD-L1⁺ cell (low risk in light gray; high risk in dark gray), with the associated patient stratification chart at the bottom. “Time” on the X-axis illustrates days of progression-free survival after treatment.

### DETAILED DESCRIPTION OF THE INVENTION

### I. Abbreviations and Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The terms “comprise,” “comprises,” and “comprising,” when preceding the recitation of a step or an element, are intended to mean that the addition of further steps or elements is optional and not excluded.

Antibody: The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

Antibody fragment: An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)²; diabodies; linear antibodies; single-chain antibody molecules (e.g. scFv); and multispecific antibodies formed from antibody fragments.

Biomarker: As used herein, the term “biomarker” shall refer to any molecule or group of molecules found in a biological sample that can be used to characterize the biological sample or a subject from which the biological sample is obtained. For example, a biomarker may be a molecule or group of molecules whose presence, absence, or relative abundance is characteristic of a particular cell or tissue type or state; or characteristic of a particular pathological condition or state; or indicative of the severity of a pathological condition, the likelihood of progression or regression of the pathological condition, and/or the likelihood that the pathological condition will respond to a particular treatment. As another example, the biomarker may be a cell type or a microorganism (such as a bacterium, mycobacterium, fungus, virus, and the like), or a substituent molecule or group of molecules thereof.

Biomarker-specific reagent: A specific detection reagent that is capable of specifically binding directly to one or more biomarkers in the cellular sample, such as a primary antibody.

Cellular sample: As used herein, the term “cellular sample” refers to any sample containing intact cells, such as cell cultures, bodily fluid samples or surgical specimens taken for pathological, histological, or cytological interpretation.

Detection reagent: A “detection reagent” is any reagent that is used to deposit a stain in proximity to a biomarker-specific reagent in a cellular sample. Non-limiting examples include biomarker-specific reagents (such as primary antibodies), secondary detection reagents (such as secondary antibodies capable of binding to a primary antibody), tertiary detection reagents (such as tertiary antibodies capable of binding to secondary antibodies), enzymes directly or indirectly associated with the biomarker specific reagent, chemicals reactive with such enzymes to effect deposition of a fluorescent or chromogenic stain, wash reagents used between staining steps, and the like.

Detectable moiety: A molecule or material that can produce a detectable signal (such as visually, electronically or otherwise) that indicates the presence (i.e. qualitative analysis) and/or concentration (i.e. quantitative analysis) of the detectable moiety deposited on a sample. The term “detectable moiety” includes, but is not limited to, chromogenic, fluorescent, phosphorescent, and luminescent molecules and materials, catalysts (such as enzymes) that convert one substance into another substance to provide a detectable difference (such as by converting a colorless substance into a colored substance or vice versa, or by producing a precipitate or increasing sample turbidity), and labels compatible with mass cytometry imaging (such as multiplexed ion beam imaging (“MIBI,” described at Baharlou, Bodenmiller, and Ptacek) or Imaging Mass Cytometry (“IMB,” described by Baharlou and Bodenmiller)). In some examples, the detectable moiety is a fluorophore, which belongs to several common chemical classes including coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, OR, ThermoFisher Scientific, 11^{th} Edition. In other embodiments, the detectable moiety is a molecule detectable via brightfield microscopy, such as dyes including diaminobenzidine (DAB), 4-(dimethylamino) azobenzene-4′-sulfonamide (DABSYL), tetramethylrhodamine (DISCOVERY Purple), N,N′-biscarboxypentyl-5,5′-disulfonato-indo-dicarbocyanine (Cy5), and Rhodamine 110 (Rhodamine). In yet other embodiments, the detectable label is compatible with mass cytometry imaging, such as a stable metal isotope (including but lanthanide series metals).

Feature metric: A value indicative of an amount of a feature in a sample or a relationship between features in a sample. Examples include: number of cells positive for a biomarker, density of a specific cell type in a particular region, (for example, number of biomarker-positive cells over an area of an ROI, number of biomarker-positive cells over a linear distance of an edge defining an ROI, and the like), pixel density (i.e. number of biomarker-positive pixels over an area of an ROI, number of biomarker-positive pixels over a linear distance of an edge defining an ROI, and the like), mean or median distance between cells expressing biomarker(s), et cetera. A feature metric can be a total metric or a global metric.

Histochemical detection: A process involving labelling biomarkers or other structures in a tissue sample with biomarker-specific reagents and detection reagents in a manner that permits microscopic detection of the biomarker or other structures in the context of the cross-sectional relationship between the structures of the tissue sample. Examples include immunohistochemistry (IHC), chromogenic in situ hybridization (CISH), fluorescent in situ hybridization (FISH), silver in situ hybridization (SISH), and hematoxylin and eosin (H&E) staining of formalin-fixed, paraffin-embedded tissue sections.

Immune checkpoint molecule: A protein expressed by an immune cell whose activation down-regulates a cytotoxic T-cell response. Examples include PD-1, TIM-3, LAG-4, and CTLA-4.

Immune escape biomarker: A biomarker expressed by a tumor cell that helps the tumor avoid a T-cell mediated immune response. Examples of immune escape biomarkers include PD-L1, PD-L2, and IDO.

Immunological biomarker: A biomarker that is characteristic of or impacts upon an immune response to an abnormal cell, including but not limited to biomarkers that: are indicative of a particular class of immune cell (such as a CD3), characterize an immune response (such as the presence, absence, or amount of cytokine proteins or particular immune cell subtype(s)), or that are expressed by, presented by, or otherwise located on non-immune cell structure that affect the type or extent of responses of immune cell (such as cell surface expressed antigens, MHC-ligand complexes, and immune escape biomarkers).

Monoclonal antibody: An antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, or a combination thereof.

Multiplex histochemical stain: A histochemical staining method in which multiple biomarker-specific reagents that bind to different biomarkers are applied to a single section and stained with different color stains.

PD-1 axis directed therapy: A therapeutic agent that disrupts the ability of PD-1 to down-regulate T-cell activity. Examples include PD-1-specific antibodies (such as nivolumab, pembrolizumab, cemiplimab, tislelizumab, spartalizumab, MEDI0680 (AstraZeneca), toripalimab, sintilimab, cetrelimab, and pidilizumab), PD-L1-specific antibodies (such as atezolizumab, durvalumab, and avelumab), PD-1-directed bispecifics (such as tebotelimab (a PD-1/LAG3 bispecific DART® molecule); PD-1 ligand fragments and fusion proteins (such as AMP-224 (a fusion between the extracellular domain of PD-L2 and the Fc region of human IgG1)), PD-L1-directed bispecifics (such as FS118 (a PD-L1/LAG3 bispecific tetravalent antibody (F-Star Therapeutics)), and small molecule inhibitors (such as CA-170 (small molecule with binding specificity for PD-L1, PD-L2 and VISTA), and BMS-1001 & BMS-1166 (small molecules predicted to dimerize PD-L1, see, e.g., WO2015034820 & WO2015160641).

Sample: As used herein, the term “sample” shall refer to any material obtained from a subject capable of being tested for the presence or absence of a biomarker.

Secondary detection reagent: A specific detection reagent capable of specifically binding to a biomarker-specific reagent.

Section: When used as a noun, a thin slice of a tissue sample suitable for microscopic analysis, typically cut using a microtome. When used as a verb, the process of generating a section.

Serial section: As used herein, the term “serial section” shall refer to any one of a series of sections cut in sequence by a microtome from a tissue sample. For two sections to be considered “serial sections” of one another, they do not necessarily need to be consecutive sections from the tissue, but they should generally contain sufficiently similar tissue structures in the same spatial relationship, such that the structures can be matched to one another after histological staining.

Simplex histochemical stain: A histochemical staining method in which a single biomarker-specific reagent is applied to a single section and stained with a single color stain.

Specific detection reagent: Any composition of matter that is capable of specifically binding to a target chemical structure in the context of a cellular sample. As used herein, the phrase “specific binding,” “specifically binds to,” or “specific for” or other similar iterations refers to measurable and reproducible interactions between a target and a specific detection reagent, which is determinative of the presence of the target in the presence of a heterogeneous population of molecules including biological molecules. For example, an antibody that specifically binds to a target is an antibody that binds this target with greater affinity, avidity, more readily, and/or with greater duration than it binds to other targets. In one embodiment, the extent of binding of a specific detection reagent to an unrelated target is less than about 10% of the binding of the antibody to the target as measured, e.g., by a radioimmunoassay (RIA). In certain embodiments, a biomarker-specific reagent that specifically binds to a target has a dissociation constant (Kd) of ≤1 μM, ≤100 nM, ≤10 nM, ≤1 nM, or ≤0.1 nM. In another embodiment, specific binding can include, but does not require exclusive binding. Exemplary specific detection reagents include nucleic acid probes specific for particular nucleotide sequences; antibodies and antigen binding fragments thereof; and engineered specific binding compositions, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, CA), dAbs (scaffold based on VH or VL antibody domain; GlaxoSmithKline PLC, Cambridge, UK), DARPins (scaffold based on Ankyrin repeat proteins; Molecular Partners AG, Zürich, CH), ANTICALINs (scaffold based on lipocalins; Pieris AG, Freising, DE), NANOBODYs (scaffold based on VHH (camelid Ig); Ablynx N/V, Ghent, BE), TRANS-BODYs (scaffold based on Transferrin; Pfizer Inc., New York, NY), SMIPs (Emergent Biosolutions, Inc., Rockville, MD), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on Discovery Research and Clinical Validation, Current Pharmaceutical Biotechnology, Vol. 9, pp. 502-509 (2008), the content of which is incorporated by reference.

Stain: When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

Subject: As used herein, the term “subject” or “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

Test sample: A tumor sample obtained from a subject having an unknown outcome at the time the sample is obtained.

Tissue sample: As used herein, the term “tissue sample” shall refer to a cellular sample that preserves the cross-sectional spatial relationship between the cells as they existed within the subject from which the sample was obtained.

Tumor mutational burden: Quantification of total number of nonsynonymous mutations per coding area of a tumor genome.

Tumor sample: A tissue sample obtained from a tumor.

### II. Biomarker Descriptions

CD3: CD3 is a cell surface receptor complex that is frequently used as a defining biomarker for cells having a T-cell lineage. The CD3 complex is composed of 4 distinct polypeptide chains: CD3-gamma chain, CD3-delta chain, CD3epsilon chain, and CD3-zeta chain. CD3-gamma and CD3-delta each form heterodimers with CD3-epsilon (εγ-homodimer and &d-heterodimer) while CD3-zeta forms a homodimer (ζζ-homodimer). Functionally, the εγ-homodimer, εδ-heterodimer, and ζζ-homodimer form a signaling complex with T-cell receptor complexes. Exemplary sequences for (and isoforms and variants of) the human CD3-gamma chain, CD3-delta chain, CD3epsilon chain, and CD3-zeta chain can be found at Uniprot Accession Nos. P09693 (the canonical amino acid sequence for which is disclosed herein at SEQ ID NO: 1), P04234 (the canonical amino acid sequence for which is disclosed herein at SEQ ID NO: 2), P07766 (the canonical amino acid sequence for which is disclosed herein at SEQ ID NO: 3), and P20963 (the canonical amino acid sequence for which is disclosed herein at SEQ ID NO: 4), respectively. As used herein, the term “human CD3 protein biomarker” encompasses any CD3-gamma chain, CD3-delta chain, CD3epsilon chain, and CD3-zeta chain polypeptide having a canonical human sequence and natural variants thereof that maintain the function of the canonical sequence; εγ-homodimers, εδ-heterodimers, and ζζ-homodimers including one of more of CD3-gamma chain, CD3-delta chain, CD3epsilon chain, and CD3-zeta chain polypeptide having a canonical human sequence and natural variants thereof that maintain the function of the canonical sequence; and any signaling complex including one or more of the foregoing CD3 homodimers or heterodimers. In some embodiments, a human CD3 protein biomarker-specific agent encompasses any biomarker-specific agent that specifically binds a structure (such as an epitope) within CD3-gamma chain polypeptide (such as the polypeptide at SEQ ID NO: 1), CD3-delta chain polypeptide (such as the polypeptide at SEQ ID NO: 2), CD3epsilon chain polypeptide (such as the polypeptide at SEQ ID NO: 3), or CD3-zeta chain polypeptide (such as the polypeptide at SEQ ID NO: 4), or that binds to a structure (such as an epitope) located within εγ-homodimer, εδ-heterodimer, or ζζ-homodimer.

CD8: CD8 is a heterodimeric, disulphide linked, transmembrane glycoprotein found on the cytotoxic-suppressor T cell subset, on thymocytes, on certain natural killer cells, and in a subpopulation of bone marrow cells. Exemplary sequences for (and isoforms and variants of) the human alpha- and beta-chain of the CD8 receptor can be found at Uniprot Accession Nos. P01732 (the canonical amino acid sequence for which is disclosed herein at SEQ ID NO: 5) and P10966 (the canonical amino acid sequence for which is disclosed herein at SEQ ID NO: 6), respectively. As used herein, the term “human CD8 protein biomarker” encompasses any CD8-alpha chain polypeptide having a canonical human sequence and natural variants thereof that maintain the function of the canonical sequence; any CD8-beta chain polypeptide having a canonical human sequence and natural variants thereof that maintain the function of the canonical sequence; any dimers including a CD8-alpha chain polypeptide having a canonical human sequence and natural variants thereof that maintain the function of the canonical sequence and/or a CD8-beta chain polypeptide having a canonical human sequence and natural variants thereof that maintain the function of the canonical sequence. In some embodiments, a human CD8 protein biomarker-specific agent encompasses any biomarker-specific agent that specifically binds a structure (such as an epitope) within CD8-alpha chain polypeptide (such as the polypeptide at SEQ ID NO: 5), CD8-beta chain polypeptide (such as the polypeptide at SEQ ID NO: 6), or that binds to a structure (such as an epitope) located within a CD8 dimer.

CD68: CD68 is a glycoprotein encoded by the CD68 gene located on chromosome 17 at location 17p13.1. CD68 protein is found in the cytoplasmic granules of a variety of different blood cells and myocytes, and is frequently used as a biomarker for cells of macrophage lineage, including monocytes, histiocytes, giant cells, Kupffer cells, and osteoclasts. Exemplary sequences for (and isoforms and variants of) human CD68 can be found at Uniprot Accession No. P34810 (the canonical amino acid sequence for which is disclosed herein at SEQ ID NO: 7). As used herein, the term “human CD68 protein biomarker” encompasses any CD68 polypeptide having a canonical human sequence and natural variants thereof that maintain the function of the canonical sequence. In some embodiments, a human CD20 protein biomarker-specific agent encompasses any biomarker-specific agent that specifically binds a structure (such as an epitope) within a human CD68 polypeptide (such as the polypeptide at SEQ ID NO: 7).

Pancytokeratin: As used herein, “pancytokeratin” and “PanCK” refer to any biomarker-specific reagent or group of biomarker-specific reagents that specifically bind to a sufficient plurality of cytokeratins to specifically stain epithelial tissue in a tissue sample. Exemplary pancytokeratin biomarker-specific reagents typically include either: (a) a single cytokeratin-specific reagent that recognizes an epitope common to the plurality of cytokeratins, wherein most epithelial cells of the tissue express at least one of the plurality of cytokeratins; or (b) a cocktail of a biomarker-specific reagents such that the cocktail is specifically reactive with a plurality of cytokeratins, wherein most epithelial cells of the tissue express at least one of the plurality of cytokeratins. Reference to a “cocktail” in this definition includes both a single composition comprising each member of the plurality, or providing each member of the plurality as separate compositions, but staining them with a single dye, or combinations thereof. PanCK cocktails are reviewed by NordiQC. In some embodiments, the PanCK biomarker-specific reagent includes antibody cocktails containing two or more of antibody clones selected from the group consisting of 5D3, LP34, AE1, AE2, AE3, MNF116, and PCK-26. In an embodiment, a PanCK cocktail is selected from the group consisting of: a cocktail of AE1 & AE3, a cocktail of AE1, AE3, and 5D3, and a cocktail of AE1, AE3, and PCK26. Cocktails of AE1 & AE3 are commercially available from Agilent Technologies (Cat. Nos. GA05361-2, IS05330-2, IR05361-2, M351501-2 and M351529-2). Cocktails of AE1, AE3, and 5D3 are commercially available from BioCare (Cat. Nos. CM162, IP162, OAI162, and PM162) and Abcam (Cat. No. ab86734). Cocktails of AE1, AE3, and PCK26 are available from Roche (Cat. No. 760-2135).

PD-1: Programmed death-1 (PD-1) is a member of the CD28 family of receptors encoded by the PDCD1 gene on chromosome 2. Exemplary sequences for (and isoforms and variants of) the human PD-1 protein can be found at Uniprot Accession No. Q15116 (the canonical amino acid sequence for which is disclosed herein at SEQ ID NO: 8). In some embodiments, a human PD-1 protein biomarker-specific agent encompasses any biomarker-specific agent that specifically binds a structure (such as an epitope) within a human PD-1 polypeptide (such as the polypeptide at SEQ ID NO: 8).

PD-L1: Programmed death ligand 1 (PD-L1) is a type 1 transmembrane protein encoded by the CD274 gene on chromosome 9. PD-L1 acts as a ligand for PD-1 and CD80. Exemplary sequences for (and isoforms and variants of) the human PD-L1 protein can be found at Uniprot Accession No. Q9NZQ7 (the canonical amino acid sequence for which is disclosed herein at SEQ ID NO: 9). In some embodiments, a human PD-L1 protein biomarker-specific agent encompasses any biomarker-specific agent that specifically binds a structure (such as an epitope) within a human PD-L1 polypeptide (such as the polypeptide at SEQ ID NO: 9).

### III. Predicting Response to PD-1 Axis-Directed Therapies

Tissue-based biomarker signatures have potential to stratify dMMR/MSI-H metastatic CRC (mCRC) subjects based on their likelihoods of gaining survival benefit from anti-PD-1 therapy. Incorporating such biomarker signatures into clinical practice may not only optimize therapeutic outcome, but also spare patients from unnecessary adverse events.

Disclosed herein are methods for predicting response to PD-1 axis-directed therapies in colorectal tumors that are one or more of dMMR and MSI-H for which PD-1 axis-directed therapy is being considered, including stage III and stage IV tumors.

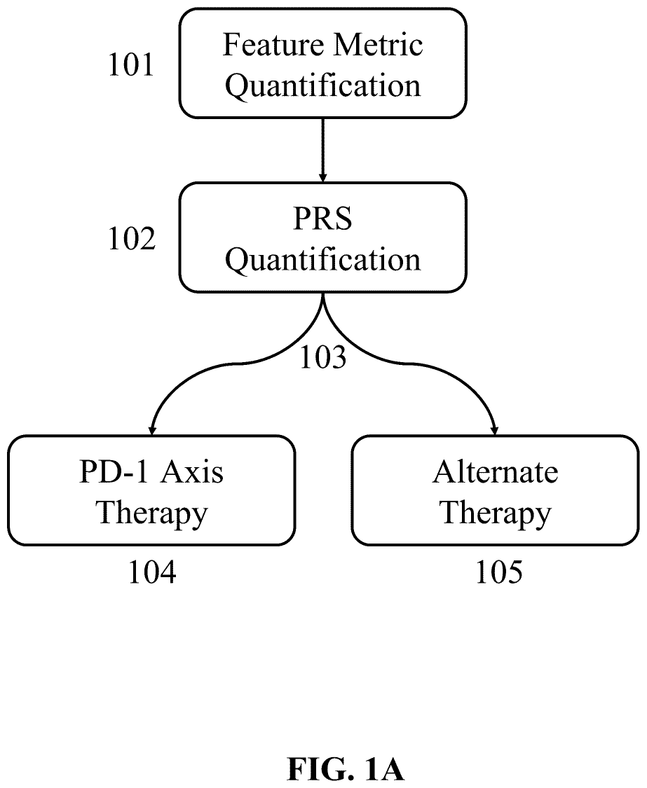

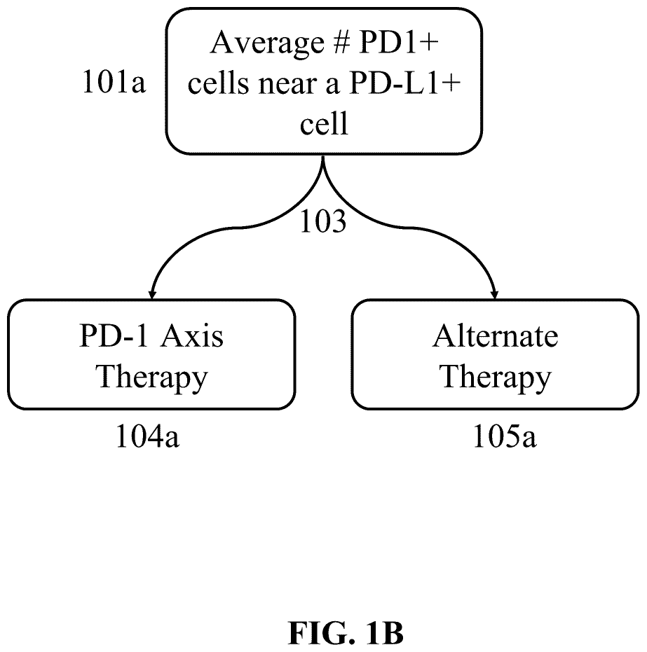

An overview of one exemplary process is outlined at FIG. 1A. One or more sets of feature metrics are derived from one or more affinity histochemistry (AHC) labeled samples of the tumor (hereafter referred to as a “Feature Set”) 101. A scoring function integrates the feature metrics (optionally including other variables) into a predicted response score (PRS) that correlates with the likelihood that the subject will respond to the PD-1 axis directed therapy 102 and compared to one or more pre-determined cutoffs. In some embodiments, the PRS is indicative of the likelihood overall survival (OS) after receiving the PD-1 axis directed therapy. In another embodiment, the PRS is indicative of the likelihood of progression-free survival (PFS) after receiving the PD-1 axis directed therapy. Depending on the PRS, the subject is either administered the PD-1 axis-directed therapy 104 or is administered an alternate therapy 105.

An overview of another exemplary process is provided at FIG. 1B. In this example, the feature metric 101 is the average number of PD-1⁺ cells within a pre-determined distance of at least 1 PD-L1⁺ cell. The PRS is a univariate analysis comparing the average number of PD-1⁺ cells within the pre-determined distance of at least 1 PD-L1⁺ cell to a pre-determined cutoff that correlates with the likelihood that the subject will respond to the PD-1 axis directed therapy 103. If the average number exceeds the pre-determined cutoff, the subject is administered the PD-1 axis-directed therapy 104a. If the average number falls below the pre-determined cutoff, the alternate therapy is administered 105a.

### III.A. Feature Set Quantification

The Feature Sets useful in the present methods are selected from the group consisting of Feature Set 1, Feature Set 2, Feature Set 3, Feature Set 4, and Feature Set 5 as set forth in Table 1 (FS: Feature Set; SD: standard deviation; MnD: mean distance; MdD: median distance; PTO: Peritumor outer; EM: epithelial marker):

TABLE 1

| FS | No. | Features | Feature Metrics | ROI |
|---|---|---|---|---|
| 1 | A | 1. CD8⁺ cell | 1. SD of distance of CD8+ cells from | Stroma |
| | | 2. CD68⁺/PDL1⁺ cell | CD68+/PDL1+ cells within 10 μm | |
| | B | 1. CD8⁺/PD-1⁺ cell | 2. MnD of CD8+/PD-1+ cells from | Tumor |
| | | 2. CD8⁺/PD-L1⁺ cell | CD8+/PD-L1+ cells within 30 μm | |
| 2 | A | 1. CD8⁺ cell | 3. SD of distance of CD8+ cells from | PTO |
| | | 2. CD68⁺/PDL1⁺ cell | CD68+/PDL1+ cells within 10 μm | |
| | B | 1. CD8⁺ cell | 4. Density of CD8+ cells | Tumor |
| 3 | A | 1. CD8⁺ cell | MdD CD8+ cells from EM+ cells | Tumor |
| | | 2. EM⁺ cell | within 30 μm | |
| | B | 1. CD8⁺ cell | MnD of CD8+ cells from PD-L1+/epithelial | Stroma |
| | | 2. PD-L1⁺/EM⁺ cell | marker+ (EM+) cells within 10 μm | |
| | C | 1. CD8⁺/PD1⁺ cell | Mean number CD8+/PD-1+ cells | Tumor |
| | | 2. CD8⁺/PDL1⁺ cell | within 10 μm of CD8+/PD-L1+ cells | |
| | | | MnD of CD8+/PD-1+ cells from | |
| | | | CD8+/PD-L1+ cells within 30 μm | |

| | | | |
|---|---|---|---|
| 4 | 1. CD8⁺ cell | Density of CD8⁺ | Tumor |
| 5 | 1. PD-1⁺ cell | Average number of PD-1+ cells within a | Tumor |
| | 2. PD-L1⁺ cell | pre-determined distance of a PD-L1+ cell | |

For Feature Set 5, the pre-determined distance is generally in the range of 5-50 μm, including, for example, 5, 10, 15, 20, 25, 30, 35, 40, 45, and 50 μm. In a specific embodiment, the pre-determined distance for Feature Set 5 is 10 μm.

These Feature Sets are extracted from one or more tissue samples from the tumor that have been labeled for the indicated biomarker(s) by an affinity histochemical (AHC) assay. Each Feature Set includes (a) a set of features to be identified in the sample (such as a cell type categorized by the status of one or biomarkers); (b) a metric describing a spatial relation between one or more of the features (such as a density of a feature or a distance between two features); and (c) a region of interest (ROI) from which the metric is described.

### III.A.1. Samples

The samples used for the AHC assay are typically tissue samples processed in a manner compatible with histochemical labeling, including, for example, fixation, embedding in a wax matrix (such as paraffin), and sectioning (such as with a microtome). No specific processing step is required by the present disclosure, so long as the sample obtained is compatible with multiplex histochemical labeling of the sample for the biomarkers of interest, generating a digital image of the labeled sample, and identification of the regions of interest in which the features are identified. In a specific embodiment, the sample is a microtome section of a formalin-fixed, paraffin-embedded (FFPE) sample.

The samples are from tumors previously determined to have one or more of dMMR or MSI-H. In a specific embodiment, the tumor is a stage III tumor. In another specific embodiment, the tumor is a stage IV tumor.

Mismatch repair status (also termed “MMR”) typically involves evaluating the expression and/or methylation status of four genes involved in mismatch repair: hPMS2, hMLH1, hMSH2, and hMSH6. A tumor having deficient expression of any one of these four is determined to have deficient mismatch repair (termed “dMMR”), while a tumor that is not deficient in expression of any of these genes is determined to have proficient MMR (termed “pMMR”). MMR status may be determined, for example, a protein-based assay (such as by immunoassay, such as a solid-phase enzyme immunoassay (e.g., ELISA) or affinity histochemical assay (AHC) assay) or a polymerase chain reaction (PCR) assay (such as a real-time reverse transcriptase PCR assay).

A microsatellite instable (“MSI”) tumor is a tumor in which alterations in the length of microsatellite loci have accumulated in the tumor beyond a pre-determined threshold. In contrast, a microsatellite stable (MSS) tumor has not accumulated alterations in the length of microsatellite loci beyond the pre-determined threshold. Assays for evaluating MSI/MSS status are well known in the art. See, e.g., Murphy et al., J. Mol. Diagn., Vol. 8, Issue 3, pp. 305-11 (July 2006); Esemuede et al., Ann. Surg. Oncol., vol. 17, Issue 12, pp. 3370-78 (December 2010); Mukherjee et al., Hereditary Cancer in Clinical Practice, Vol. 8, Issue 9 (2010); MSI Analysis System (Promega) (evaluation of seven markers for MSI phenotype, including five nearly monomorphic mononucleotide repeat markers (BAT-25, BAT-26, MONO-27, NR-21 and NR-24) and two highly polymorphic pentanucleotide repeat markers (Penta C and Penta D)).

### III.A.2. AHC Panels

The AHC assays rely on one or more panels of biomarker-specific reagents. In an embodiment, at least one of the panels is a multiplex panel. As used in the context of this disclosure, a “multiplex panel” shall refer to a set of biomarker specific reagents that are useful in a multiplex AHC (“mAHC”) assay to differentially label multiple biomarkers in the same sample. At a minimum, the multiplex panels must be sufficient to label a tissue in a manner that permits detection of at least one of the features and measurement of at least one of the feature metrics of Feature Sets 1-4.

A single multiplex panel may be used to label all of the features and detect all of the feature metrics of the respective feature set. Exemplary multiplex panels useful for such an embodiment are set forth in Table 2 (* indicates an optional biomarker-specific reagent):

TABLE 2

| Panel No. | Multiplex Panel | Feature Set |
|---|---|---|
| A | CD8, CD68, PD-L1, PD-1, EM* | 1 |
| B | CD8, CD68, PD-L1, EM* | 2 |
| C | CD8, EM*, PD-L1, PD-1 | 3 |
| D | CD8, EM | 4 |
| E | PD-1, PD-L1 | 5 |

Alternatively, separate panels may be used for the different feature metrics of the respective Feature Set. Exemplary panels for Feature Set 1-3 are set forth at Tables 3-6 (* indicates an optional biomarker-specific reagent):

TABLE 3
FEATURE SET 1

| Panel | Biomarkers | Features | Feature Metric |
|---|---|---|---|
| B | 1. CD8 | 1. CD8+ cells | SD of distance of CD8+ |
| | 2. CD68 | 2. CD68+/PDL1+ | cells from CD68+/PDL1+ |
| | 3. PD-L1 | cells | cells within 10 μm |
| | 4. EM* | | |
| C | 1. CD8 | 1. CD8+/PD-1+ | MD of CD8+/PD-1+ |
| | 2. PD-L1 | cells | cells from CD8+/PD-L1+ |
| | 3. PD-1 | 2. CD8+/PD-L1+ | cells within 30 μm |
| | 4. EM* | cells | |

TABLE 4
FEATURE SET 2

| Panel | Biomarkers | Features | Feature Metric |
|---|---|---|---|
| B | 1. CD8 | 1. CD8+ cells | SD of distance of CD8+ |
| | 2. CD68 | 2. CD68+/PDL1+ | cells from CD68+/PDL1+ |
| | 3. PD-L1 | cells | cells within 10 μm |
| | 4. EM* | | |
| D | 1. CD8 | 1. CD8+ cells | Density of CD8+/EM− |
| | 2. EM* | | cells |

TABLE 5
FEATURE SET 3 (Example 1)

| Panel | Biomarkers | Features | Feature Metric |
|---|---|---|---|
| D | 1. CD8 | 1. CD8+ cell | MdD CD8+ cells from |
| | 2. EM | 2. EM+ cell | EM+cells w/in 30 μm |
| F | 1. CD8 | 1. CD8+ cell | MnD of CD8+ cells from |
| | 2. PD-L1 | 2. PD-L1+/EM+ | PD-L1+/EM+ cells |
| | 3. EM | cell | within 10 μm |
| E | 1. CD8 | 1. CD8+/PD-1+ | 1. Mean number CD8+/ |
| | 2. PD-1 | cell | PD-1+cells from CD8+/ |
| | 3. PD-L1 | 2. CD8+/PD-L1+ | PD-L1+ cells within 10 μm |
| | 4. EM* | cell | 2. MnD from CD8+/PD-1+ |
| | | | cells to CD8+/PD-L1+ |
| | | | cells w/in 30 μm |

TABLE 6
FEATURE SET 3 (Example 2)

| Panel | Biomarkers | Features | Feature Metric |
|---|---|---|---|
| D | 1. CD8 | 1. CD8+ cell | MdD CD8+ cells from EM+ |
| | 2. EM | 2. EM+ cell | cells w/in 30 μm |
| C | 1. CD8 | 1. CD8+ cell | 1. MnD of CD8+ cells from |
| | 2. PD-1 | 2. PD-L1+/EM− | PD-L1+/EM+ cells within 10 |
| | 3. PD-L1 | cell | μm |
| | 4. EM* | 3. CD8+/PD-1+ | 2. Mean number CD8+/PD-1+ |
| | | cell | cells from CD8+/PD-L1+ cells |
| | | 4. CD8+/PD-L1+ | within 30 μm |
| | | cell | 2. MnD of CD8+/PD-1+ cells |
| | | | from CD8+/PD-L1+ cells w/in |
| | | | 30 μm |

TABLE 7
FEATURE SET 3 (Example 3)

| Panel | Biomarkers | Features | Feature Metric |
|---|---|---|---|
| D | 1. CD8 | 1. CD8+ cell | 1. MdD CD8+ cells from EM+ |
| | 2. EM | 2. EM+ cell | cells w/in 30 μm |
| | 3. PD-L1 | 3. PD-L1+/EM− | 2. MnD of CD8+ cells from |
| | | cell | PD-L1+/EM+ cells w/in 10 |
| | | | μm |
| B | 1. CD8 | 1. CD8+/PD-1+ | 1. Mean number CD8+/PD-1+ |
| | 2. PD-1 | cell | cells from CD8+/PD-L1+ cells |
| | 3. PD-L1 | 2. CD8+/PD-L1+ | within 10 μm |
| | 4. EM | cell | 2. MnD of CD8+/PD-1+ cells |
| | | | from CD8+/PD-L1+ cells w/in |
| | | | 30 μm |

These examples are not intended to be an exhaustive listing of panels useful to detect the features and feature metrics. In an exemplary embodiment, the AHC is a multiplex immunohistochemical (mIHC) assay comprising applying at least one of the multiplex panels selected from the group consisting of multiplex panel A-F to a tissue sample from the tumor by a mIHC method, wherein the biomarker-specific reagents of the panel are antibodies. In another embodiment, the multiplex panel selected from the group consisting of multiplex panel A-F includes the antibody against one or more epithelial markers. In yet another embodiment, the antibody against one or more epithelial markers of Panel A-F is a panCK antibody.

### III.A.3. Biomarker Labeling, Counterstaining, and Morphological Staining

The panels of biomarker-specific reagents are used in combination with a set of appropriate detection reagents to generate a biomarker-labeled section. Biomarker labeling is typically accomplished by contacting a section of the sample with a biomarker-specific reagent under conditions that facilitate specific binding between the biomarker and the biomarker-specific reagent. The sample is then contacted with a set of detection reagents that interact with the biomarker-specific reagent to facilitate deposition a detectable moiety in close proximity the biomarker, thereby generating a detectable signal localized to the biomarker. Typically, wash steps are performed between application of different reagents to prevent unwanted non-specific labeling of tissues. Biomarker-labeled sections may optionally be additionally labeled with a contrast agent (such as a hematoxylin stain) to visualize macromolecular structures. Additionally, a serial section of the biomarker-labeled section(s) may be labeled with a morphological stain to facilitate ROI identification.

The detectable moieties used with the panels should be compatible with multiplex affinity histochemical labeling methods, such as multiplex immunohistochemistry (IHC). In some embodiments, the detectable moiety is a fluorophore. Exemplary fluorophores include several common chemical classes, such as coumarins, fluoresceins (or fluorescein derivatives and analogs), rhodamines, resorufins, luminophores and cyanines. Additional examples of fluorescent molecules can be found in Molecular Probes Handbook-A Guide to Fluorescent Probes and Labeling Technologies, Molecular Probes, Eugene, OR, ThermoFisher Scientific, 11th Edition. Exemplary fluorescent dyes compatible with multiplex IHC and methodologies of using the same are disclosed at, for example, Gorris, Hofman, and Parra, In other embodiments, the detectable moiety is a molecule detectable via brightfield microscopy. Exemplary brightfield dyes compatible with multiplex IHC and methodologies of using the same are disclosed at, for example, Hofman, Ide, Morrison, Parra, Stack, and U.S. Pat. No. 10,041,950 B2. Specific examples include diaminobenzidine (DAB), 4-(dimethylamino) azobenzene-4′-sulfonamide (DABSYL), tetramethylrhodamine (DISCOVERY Purple), N,N′-biscarboxypentyl-5,5′-disulfonato-indo-dicarbocyanine (Cy5), and Rhodamine 110 (Rhodamine). In yet other embodiments, the detectable moiety is a mass spectrometer-detectable label. Reviews of mass spectrometry-based multiplexing methods and labels can be found at Levenson and Parra, for example.

Non-limiting examples of commercially available detection reagents or kits comprising detection reagents suitable for use with present methods include: VENTANA ULTRAVIEW detection systems (secondary antibodies conjugated to enzymes, including HRP and AP); VENTANA IVIEW detection systems (biotinylated anti-species secondary antibodies and streptavidin-conjugated enzymes); VENTANA OPTIVIEW detection systems (OptiView) (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OPTIVIEW Amplification system (Anti-species secondary antibody conjugated to a hapten, an anti-hapten tertiary antibody conjugated to an enzyme multimer, and a tyramide conjugated to the same hapten. In use, the secondary antibody is contacted with the sample to effect binding to the primary antibody. Then the sample is incubated with the anti-hapten antibody to effect association of the enzyme to the secondary antibody. The sample is then incubated with the tyramide to effect deposition of additional hapten molecules. The sample is then incubated again with the anti-hapten antibody to effect deposition of additional enzyme molecules. The sample is then incubated with the detectable moiety to effect dye deposition); VENTANA DISCOVERY, DISCOVERY OMNIMAP, DISCOVERY ULTRAMAP anti-hapten antibody, secondary antibody, chromogen, fluorophore, and dye kits, each of which are available from Ventana Medical Systems, Inc. (Tucson, Arizona); POWERVISION and POWER VISION+ IHC Detection Systems (secondary antibodies directly polymerized with HRP or AP into compact polymers bearing a high ratio of enzymes to antibodies); DAKO ENVISION™+ System (enzyme labeled polymer that is conjugated to secondary antibodies); ULTRAPLEX Multiplex Chromogenic IHC Technology from CELL IDx (hapten-labeled primary antibodies combined with enzyme-labeled or fluor-labeled anti-hapten secondary antibodies).

If desired, the biomarker-labeled slides may be counterstained to assist in identifying morphologically relevant areas for identifying ROIs, either manually or automatically. Examples of counterstains include chromogenic nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green); non-nuclear chromogenic stains, such as eosin (stains pink); fluorescent nuclear stains, including 4′,6-diamino-2-pheylindole (DAPI, stains blue), propidium iodide (stains red), Hoechst stain (stains blue), nuclear green DCS1 (stains green), nuclear yellow (Hoechst S769121, stains yellow under neutral pH and stains blue under acidic pH), DRAQ5 (stains red), DRAQ7 (stains red); fluorescent non-nuclear stains, such as fluorophore-labelled phalloidin, (stains filamentous actin, color depends on conjugated fluorophore).

The AHC assay and counterstain may be applied to the sample using an automated AHC labeling system. Automated AHC labeling systems typically include at least: reservoirs of the various reagents used in the labeling protocols, a reagent dispense unit in fluid communication with the reservoirs for dispensing reagent to onto a sample, a waste removal system for removing used reagents and other waste from the sample, and a control system that coordinates the actions of the reagent dispense unit and waste removal system. In addition to performing labeling steps, many automated AHC labeling systems can also perform steps ancillary to labeling (or are compatible with separate systems that perform such ancillary steps), including: slide baking (for adhering the sample to a slide), dewaxing (also referred to as deparaffinization), antigen retrieval, counterstaining, dehydration and clearing, and coverslipping. Prichard, Overview of Automated Immunohistochemistry, Arch Pathol Lab Med., Vol. 138, pp. 1578-1582 (2014), incorporated herein by reference in its entirety, describes several specific examples of automated AHC labeling systems and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and LAB VISION AUTOSTAINER (Thermo Scientific) automated AHC labeling systems. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published patents application Nos. 20030211630 and 20040052685, each of which is incorporated herein by reference in its entirety. Commercially-available labeling units typically operate on one of the following principles: (1) open individual slide labeling, in which slides are positioned horizontally and reagents are dispensed as a puddle on the surface of the slide containing a tissue sample (such as implemented on the DAKO AUTOSTAINER Link 48 (Agilent Technologies) and INTELLIPATH (Biocare Medical) labelers); (2) liquid overlay technology, in which reagents are either covered with or dispensed through an inert fluid layer deposited over the sample (such as implemented on BENCHMARK and DISCOVERY labelers); (3) capillary gap labeling, in which the slide surface is placed in proximity to another surface (which may be another slide or a coverplate) to create a narrow gap, through which capillary forces draw up and keep liquid reagents in contact with the samples (such as the labeling principles used by DAKO TECHMATE, Leica BOND, and DAKO OMNIS labelers). Some iterations of capillary gap labeling do not mix the fluids in the gap (such as on the DAKO TECHMATE and the Leica BOND). In variations of capillary gap labeling termed dynamic gap labeling, capillary forces are used to apply sample to the slide, and then the parallel surfaces are translated relative to one another to agitate the reagents during incubation to effect reagent mixing (such as the labeling principles implemented on DAKO OMNIS slide labelers (Agilent)). In translating gap labeling, a translatable head is positioned over the slide. A lower surface of the head is spaced apart from the slide by a first gap sufficiently small to allow a meniscus of liquid to form from liquid on the slide during translation of the slide. A mixing extension having a lateral dimension less than the width of a slide extends from the lower surface of the translatable head to define a second gap smaller than the first gap between the mixing extension and the slide. During translation of the head, the lateral dimension of the mixing extension is sufficient to generate lateral movement in the liquid on the slide in a direction generally extending from the second gap to the first gap. See WO 2011-139978 A1. It has also been proposed to use inkjet technology to deposit reagents on slides. See WO 2016-170008 A1. This list of labeling technologies is not intended to be comprehensive, and any fully or semi-automated system or manual method for performing biomarker labeling may be incorporated into the present methods.

It may also desirable to morphologically stain a serial section of the biomarker-labeled section, which can be used to identify the ROIs from which scoring is conducted. Many morphological stains are known, including but not limited to, hematoxylin and eosin (H&E) stain and Lee's Stain (Methylene Blue and Basic Fuchsin). In a specific embodiment, at least one serial section of each biomarker-labeled slide is H&E stained. Any method of applying H&E stain may be used, including manual and automated methods. In an embodiment, at least one section of the sample is an H&E stained sample stained on an automated staining system. Automated systems for performing H&E staining typically operate on one of two staining principles: batch staining (also referred to as “dip 'n dunk”) or individual slide staining. Batch stainers generally use vats or baths of reagents in which many slides are immersed at the same time. Individual slide stainers, on the other hand, apply reagent directly to each slide, and no two slides share the same aliquot of reagent. Examples of commercially available H&E stainers include the VENTANA HE 600 series H&E stainers (individual slide stainer) from Roche; the DAKO COVERSTAINER from Agilent Technologies (batch stainer); the LEICA ST4020 Small Linear Stainer, LEICA ST5020 MULTISTAINER, and the LEICA ST5010 AUTOSTAINER XL series H&E stainers from Leica Biosystems Nussloch GmbH (batch stainers).

### III.A.4. Regions of Interest

Each feature metric is derived from a specific region of interest (ROI) within the sample. The ROI is a biologically relevant location of the tissue section from which relevant spatial relationships between different cell types is evaluated. Exemplary ROIs useful in the present methods are described at Table 8:

TABLE 8

| ROI | Description |
|---|---|
| Tumor area (TA) | Area delineated by a trained user (such as |
| | a pathologist) or identified by an image |
| | analysis algorithm as containing tumor cells |
| Epithelial area (EA) | Area of EM+ cell aggregates |
| Stroma area (SA) | Any area of a sample not contained |
| | within an epithelial area or tumor area |
| Peritumor inner (PI) | An area entirely inside of the tumor area |
| | extending a pre-determined distance away |
| | from the invasive front |
| Peritumor outer (PO) | An area entirely outside of the tumor area |
| | extending a pre-determined distance away |
| | from the invasive front |
| Peritumor region (PR) | An area encompassing the invasive front and |
| | extending a predetermined distance away from |
| | the invasive front into the tumor and a |
| | predetermined distance away from the invasive |
| | front toward the outside of the tumor |

Exemplary predetermined distance away from the invasive front used for the PI, PO, and PR regions includes, but is not limited to, distances in the range of about 250 μm to about 750 μm, about 400 μm to about 600 μm, or about 500 μm. As used in this context, the term “about” shall encompass any distance that is within 10% of the recited endpoints.

### III.A.5: Spatial Relationships

The feature metric quantification 101 is based on the spatial relationship of one or more cell types within a relevant ROI. Each of the features from the feature sets 1-5 are cell types categorized by the presence and/or absence of one or more biomarkers and the relevant feature metrics are spatial relationships between the various cell types. Any method of measuring the relevant feature metrics in their respective ROI may be used.

In an embodiment, the spatial relationships are automatically quantitated using an image analysis system. An exemplary image analysis system is described below at sec. IV.

### III.B: Scoring Function

After the feature metric(s) are computed for the image(s), they are input into a scoring function to calculate an Predicted Response Score (PRS) for the tumor 102. The computed PRS is compared to one or more cutoffs 103 to determine whether the subject is more likely to respond or not respond to the PD-1 axis-directed therapy. In an exemplary embodiment, the scoring function is a continuous scoring function based upon a Feature Set selected from the group consisting of Feature Set 1, Feature Set 2, Feature Set 3, and Feature Set 4. In a further embodiment, the continuous scoring function is a Cox proportional hazard model based upon a Feature Set selected from the group consisting of Feature Set 1, Feature Set 2, Feature Set 3, and Feature Set 4.

The scoring function is typically modeled on tissue sections obtained from a cohort of subjects having a tumor and known response to the PD-1 axis directed therapy. Candidate scoring function models are generated by inputting the selected feature metrics and outcome data for each member of the cohort into a modeling function. The model having the highest concordance with response is selected as the scoring function. Exemplary modeling functions include quadrant discriminant analysis (QDA), Linear discriminant analysis (LDA), Support vector machine (SVM), Artificial neural network (ANN), and Cox Proportional Hazard Modeling (COX). In an embodiment, the candidate functions are modeled only on features metrics. In other embodiments, the candidate functions include other clinical variables, such as age, sex, location of metastases, lymph node involvement, etc. In an embodiment, the model is used to correlate the feature metrics to the likelihood of progressive disease after treatment within a pre-determined timeframe (termed “progression-free survival”). In an embodiment, the model is used to correlate the feature metrics to the likelihood that the patient will survive for a pre-determined period of time after treatment versus the likelihood that the patient will die within that timeframe of any cause (termed “overall survival”).

Additionally, one or more stratification cutoffs may be selected to separate the patients into “risk bins” according to relative risk (such as “high risk” and “low risk,” quartiles, deciles, etc.). In one example, stratification cutoffs are selected using receiver operator characteristic (ROC) curves. ROC curves allow users to balance the sensitivity of the model (i.e. prioritize capturing as many “positive” or “likely to respond” candidates as possible) with the specificity of the model (i.e. minimizing false-positives for “likely to respond” candidates). In an embodiment, a cutoff is selected between likely to respond and unlikely to respond risk bins, the cutoff chosen having the sensitivity and specificity balanced. In an embodiment, stratification cutoffs differentiate between (a) patients likely to have progressive disease after treatment and (b) patients likely to have stable disease, a partial response, or a complete response to the therapy. In an embodiment, the stratification cutoffs differentiate between (a) patients likely to have progressive disease after treatment, (b) patients likely to have stable disease after treatment, and (c) patients likely to have a partial response or a complete response to the therapy. In an embodiment, the stratification cutoffs differentiate between (a) patients likely to have progressive disease or stable disease after treatment and (b) patients likely to have a partial or complete response to the therapy. In yet another embodiment, the cutoff may be a mean or median PRS.

Models may be performed using a computerized statistical analysis software suite (such as The R Project for Statistical Computing (r-project.org), SAS, MATLAB, among others).

### III.C. Treatment

The PRS 106 is used to determine whether the subject is likely to respond to treatment with a PD-1 directed therapy 107 or would likely benefit more from an alternative therapy course 108.

In an embodiment, the PD-1 axis-directed therapy course 107 is selected from the group consisting of a PD-1-specific antibody (such as nivolumab, pembrolizumab, cemiplimab, tislelizumab, spartalizumab, MEDI0680 (AstraZeneca), toripalimab, sintilimab, cetrelimab, and pidilizumab), a PD-L1-specific antibody (such as atezolizumab, durvalumab, and avelumab), a PD-1-directed bispecific (such as tebotelimab (a PD-1/LAG3 bispecific DART® molecule); a PD-1 ligand fusion protein (such as AMP-224), a PD-L1-directed bispecifics (such as FS118), and a small molecule inhibitor (such as CA-170, BMS-1001, or BMS-1166). In an embodiment, the PD-1 axis-directed therapy includes a PD-1 directed monoclonal antibody or a PD-L1-directed monoclonal antibody. In an embodiment, the PD-1 axis-directed therapy 107 comprises a therapeutic entity selected from the group consisting of nivolumab, pembrolizumab, cemiplimab, tislelizumab, spartalizumab, MEDI0680, toripalimab, sintilimab, cetrelimab, atezolizumab, durvalumab, and avelumab. In an embodiment, the PD-1 axis-directed therapy comprises pembrolizumab.

In some embodiments, the PD-1 axis-directed therapy 107 further includes a reduced course of chemotherapy. A “reduced” course of chemotherapy could include a reduction in the number of different chemotherapy agents used, the dose of one or more chemotherapy agent(s), and/or the duration of treatment with the one or more chemotherapy agent(s). A reduced course of chemotherapy may also include selection of a chemotherapy agent that has a lower toxicity profile relative to other chemotherapy agents for the treatment of CRC.

In other embodiments, the PD-1 axis-directed therapy 107 further includes another immune checkpoint-directed therapy, such as a therapy that targets CTLA-4 (such as ipilimumab or tremilumab), IDO (such as NLG919, epacadostat, BMS-986205, PF-06840003, navoximod, indoximod, NLG802, or LY3381916), TIM-3 (such as MGB453, TSR-022, Sym023, or BGBA425 (BeiGene)), LAG3 (such as relatlimab, eftilagimod alpha, ieramilimab, REGN3767, or encelimab).

In other embodiments, the PD-1 axis-directed therapy 107 further includes another immune checkpoint-directed therapy and a reduced course of chemotherapy.

In an embodiment, the alternate therapy 108 is a standard therapeutic course for stage III or stage IV colorectal cancer. Current treatment protocols typically include, in cases where tumor counts are low, surgical removal of the tumor and nearby lymph nodes along with surgical removal of the distant metastases, and adjuvant chemotherapy before and/or after surgical removal. For stage III or IV colon cancers that are not indicated for surgery, chemotherapy is typically administered as a primary treatment, optionally in combination with a targeted therapy where indicated. Some of the most commonly used regimens include: FOLFOX: leucovorin, fluorouracil (5-FU), and oxaliplatin (ELOXATIN); FOLFIRI: leucovorin, 5-FU, and irinotecan (CAMPTOSAR); CAPEOX or CAPOX: capecitabine (XELODA) and oxaliplatin; FOLFOXIRI: leucovorin, 5-FU, oxaliplatin, and irinotecan; One of the above combinations plus either a drug that targets VEGF, (bevacizumab [AVASTIN], ziv-aflibercept [ZALTRAP], or ramucirumab [CYRAMZA]), or a drug that targets EGFR (cetuximab [ERBITUX] or panitumumab [VECTIBIX]); 5-FU and leucovorin, with or without a targeted drug; Capecitabine, with or without a targeted drug; Irinotecan, with or without a targeted drug; Cetuximab alone; Panitumumab alone; Regorafenib (Stivarga) alone; Trifluridine and tipiracil (Lonsurf).

### IV. Image Analysis System

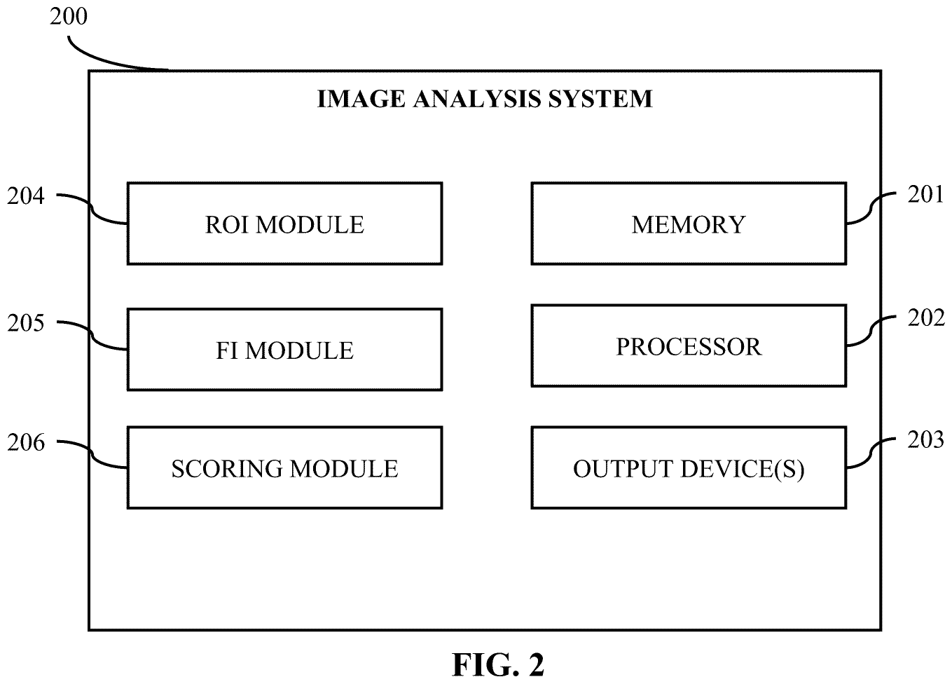

In an embodiment, Feature Metric Quantification 101 may be performed by an image analysis system. An exemplary image analysis system is illustrated at FIG. 2.

Image analysis system 200 may include one or more computing devices such as desktop computers, laptop computers, tablets, smartphones, servers, application-specific computing devices, or any other type(s) of electronic device(s) capable of performing the techniques and operations described herein. In some embodiments, image analysis system 200 may be implemented as a single device. In other embodiments, image analysis system 200 may be implemented as a combination of two or more devices together achieving the various functionalities discussed herein. For example, image analysis system 200 may include one or more server computers and a one or more client computers communicatively coupled to each other via one or more local-area networks and/or wide-area networks such as the Internet.

Image analysis system 200 includes a memory 201, a processor 202, and one or more output device(s) (such as a display, a printer, or a non-transitory storage device (such as a hard drive, flash drive, cloud drive, etc.)) 203. Memory 201 may include any combination of any type of volatile or non-volatile memories, such as random-access memories (RAMs), read-only memories such as an Electrically-Erasable Programmable Read-Only Memory (EEPROM), flash memories, hard drives, solid state drives, optical discs, and the like. For brevity purposes memory 201 is depicted in FIG. 2 as a single device, but it is appreciated that memory 201 can also be distributed across two or more devices.

Processor 202 may include one or more processors of any type, such as central processing units (CPUs), graphics processing units (GPUs), special-purpose signal or image processors, field-programmable gate arrays (FPGAs), tensor processing units (TPUs), and so forth. For brevity purposes processor 202 is depicted in FIG. 2 as a single device, but it is appreciated that processor 202 can also be distributed across any number of devices.

The processor 202 implements a set of instructions stored on the memory 201, the set of instructions comprising extracting a Feature Set from one or more digital images of an affinity histochemically (AHC) labeled tissue sample from a stage IV colorectal tumor previously determined to be one or more of dMMR or MSI-H, wherein the Feature Set is selected from the group consisting of Feature Set 1, Feature Set 2, Feature Set 3, and Feature Set 4. In an embodiment, the Feature Set is extracted by implementing a Feature Identification (FI) Module 204, a Region of Interest (ROI) Module 205, and a Scoring Module 206 on the digital image(s).

### IV.A. FI Module

The FI Module 204 functions to identify features within the image that correlate to cells and associate the identified cells with a feature vector describing the biomarker status of the cell. The output of FI Module 204 is effectively a map of the image annotating the position of all cells within the image (or a specified region of the image) with a feature vector for each marked cell with sufficient information to categorize the cell as being biomarker positive (+) or biomarker negative (−) for the relevant biomarkers.

In an embodiment, the FI Module 204 is programed to identify and categorize the objects of Feature Set 1 in a single digital image. In such an embodiment, the image analysis system is programmed to (a) mark all cells; (b) for at least the cells in a stroma region, generate a feature vector indicating CD8, CD68, and PD-L1 status; and (c) for at least the cells in the tumor region, generate a feature vector indicating CD8, PD-1, and PD-L1 status. The scoring function 206 generates feature metrics are then computed: (1) standard deviation of distance of CD8+ cells from CD68+/PDL1+ cells w/in 10 μm in a stroma region; and (2) mean distance of CD8+/PD-1+ cells from CD8+/PD-L1+ cells w/in 30 μm in a tumor region. In such an embodiment, the mAHC-labeled section of the tumor may be labeled with a first detectable moiety via a biomarker specific reagent for CD8, a second detectable moiety via a biomarker-specific reagent for CD68, a third detectable moiety via a biomarker-specific reagent for PD-L1, a fourth detectable moiety via a biomarker specific reagent for PD-1, and, optionally, a fifth detectable moiety via a biomarker-specific reagent for an EM, wherein the first, second, third, fourth, and fifth detectable moieties are distinguishable from one another when labeling the same cell.

In another embodiment, the FI Module 204 is programed to identify and categorize the objects of Feature Set 1 in separate digital images from the same tumor. In such an embodiment, the image analysis system may be programmed to execute a first FI Module 204 on a first digital image of a first mAHC-labeled section of a tumor and a second FI Module 204 on a second digital image of a second mAHC-labeled section of the tumor, wherein (a) the first FI Module 204 marks at least all the cells in the stroma region that are CD8+ status and at least all the cells in the stroma region that are CD68+/PD-L1+; and (b) the second FI Module 204 marks at least all the cells in the tumor region that are CD8+/PD-1+ and all the cells in the tumor region that are CD8+/PD-L1+. The following feature metric is computed from the first digital image: standard deviation of distance of CD8+ cells from CD68+/PDL1+ cells w/in 10 μm in a stroma region; and the following feature metric is computed from the second digital image: mean distance of CD8+/PD-1+ cells from CD8+/PD-L1+ cells w/in 30 μm in a tumor region. In such an embodiment, the first mAHC-labeled section of the tumor may be labeled with a first detectable moiety via a biomarker specific reagent for CD8, a second detectable moiety via a biomarker-specific reagent for CD68, and a third detectable moiety via a biomarker-specific reagent for PD-L1, wherein the first, second, and third detectable moieties are distinguishable from one another when labeling the same cell; and the second mAHC-labeled section of the tumor may be labeled with a fourth detectable moiety via a biomarker specific reagent for CD8, a fifth detectable moiety via a biomarker-specific reagent for PD-1, and a sixth detectable moiety via a biomarker-specific reagent for PD-L1, wherein the fourth, fifth, and sixth detectable moieties (which may be the same or different from the first, second, and third detectable moieties) are distinguishable from one another when labeling the same cell.

In another embodiment, the FI Module 204 is programed to identify and categorize the objects of Feature Set 2 in a single digital image. In such an embodiment, the image analysis system is programmed to (a) for at least the cells in a peritumor outer region, mark all cells that are CD8+ cells and all of the cells that are CD68+/PDL1+ cells; and (b) for at least the cells in the tumor region, mark all cells that are CD8+. The following feature metrics are then computed: (1) standard deviation of distance of CD8+ cells from CD68+/PDL1+ cells within 10 μm in a peritumor outer region; and (2) density of CD8+ cells in a tumor region. In such an embodiment, the mAHC-labeled section of the tumor may be labeled with a first detectable moiety via a biomarker specific reagent for CD8, a second detectable moiety via a biomarker-specific reagent for CD68, a third detectable moiety via a biomarker-specific reagent for PD-L1, and, optionally, a fourth detectable moiety via a biomarker-specific reagent for an EM, wherein the first, second, third, and fourth detectable moieties are distinguishable from one another when labeling the same cell.

In another embodiment, the FI Module 204 is programed to identify and categorize the objects of Feature Set 2 in separate digital images from the same tumor. In such an embodiment, the image analysis system may be programmed to execute a first FI Module 204 on a first digital image of a first mAHC-labeled section of a tumor and a second FI Module 204 on a second digital image of a second mAHC-labeled section of the tumor, wherein (a) the first FI Module 204 marks, at least in a peritumor outer region, all of the cells that are CD8+ and all of the cells in the peritumor outer region that are CD68+/PD-L1+; and (b) the second FI Module 204 marks at least all the cells in a tumor region that are CD8+. The following feature metric is then computed from the first digital image: standard deviation of distance of CD8+ cells from CD68+/PDL1+ cells within 10 μm in the peritumor outer region; and the following feature metric is computed from the second digital image: density of CD8+ cells in the tumor region. In such an embodiment, the first mAHC-labeled section of the tumor may be labeled with a first detectable moiety via a biomarker specific reagent for CD8, a second detectable moiety via a biomarker-specific reagent for CD68, and a third detectable moiety via a biomarker-specific reagent for PD-L1, and optionally a fourth detectable moiety via a biomarker-specific reagent for an EM marker, wherein the first, second, third, and fourth detectable moieties are distinguishable from one another when labeling the same cell; and the second mAHC-labeled section of the tumor may be labeled with a fifth detectable moiety via a biomarker specific reagent for CD8, and optionally a sixth detectable moiety via a biomarker-specific reagent for and EM marker, wherein the fifth and sixth detectable moieties (which may be the same or different from any of the first, second, third, and fourth detectable moieties) are distinguishable from one another when labeling the same cell.

In another embodiment, the FI Module 204 is programed to identify and categorize the objects of Feature Set 3 in a single digital image. In such an embodiment, the image analysis system is programmed to (a) for at least the cells in a tumor region, mark all cells that are CD8+, all cells that are CD8+/PD-1+, all cells that are CD8+/PD-L1+, and all cells that are EM+ cells; and (b) for at least the cells in a stroma region, mark all cells that are CD8+ and all cells that are PD-L1+/EM+. The following feature metrics are then computed: (1) median distance of CD8+ cells from EM+ cells w/in 30 μm in the tumor region; (2) median distance from CD8+ cells to PD-L1+/epithelial marker+ (EM+) cells within 10 μm in the stroma region; (3) mean number of CD8+/PD-1+ cells within 10 μm of CD8+/PD-L1+ cells; and (4) median distance from CD8+/PD-1+ cells to CD8+/PD-L1+ cells within 30 μm. In such an embodiment, the mAHC-labeled section of the tumor may be labeled with a first detectable moiety via a biomarker specific reagent for CD8, a second detectable moiety via a biomarker-specific reagent for PD-L1, a third detectable moiety via a biomarker-specific reagent for PD-1, and a fourth detectable moiety via a biomarker-specific reagent for an EM, wherein the first, second, third, and fourth detectable moieties are distinguishable from one another when labeling the same cell.

In another embodiment, the FI Module 204 is programed to identify and categorize the objects of Feature Set 3 in separate digital images from the same tumor. In one such an embodiment, the image analysis system may be programmed to execute a first FI Module 204 on a first digital image of a first mAHC-labeled section of a tumor and a second FI Module 204 on a second digital image of a second mAHC-labeled section of the tumor, wherein (a) the first FI Module 204 marks, at least in a tumor region, all of the cells that are CD8+ and all cells that are EM+; and (b) the second FI Module 204 marks at least: (b1) all the cells in a stroma region that are CD8+, (b2) all cells in the stroma region that are PD-L1+/EM+, (b3) all cells in a tumor region that are CD8+/PD-1+, and (b4) all cells in a tumor region that are CD8+/PD-L1+. The following feature metric is then computed from the first digital image: (1) median distance of CD8+ cells from EM+ cells w/in 30 μm in the tumor region; and the following feature metrics are computed from the second digital image: (2) median distance from CD8+ cells to PD-L1+/epithelial marker+ (EM+) cells within 10 μm in the stroma region, (3) mean number of CD8+/PD-1+ cells within 10 μm of CD8+/PD-L1+ cells, and (4) median distance from CD8+/PD-1+ cells to CD8+/PD-L1+ cells within 30 μm. In such an embodiment, the first mAHC-labeled section of the tumor may be labeled with a first detectable moiety via a biomarker specific reagent for CD8 and a second detectable moiety via a biomarker-specific reagent for EM, wherein the first and second detectable moieties are distinguishable from one another when labeling the same cell; and the second mAHC-labeled section of the tumor may be labeled with a third detectable moiety via a biomarker specific reagent for CD8, a fourth detectable moiety via a biomarker-specific reagent for PD-L1, a fifth detectable moiety via a biomarker-specific reagent for PD-1, and a sixth detectable moiety via a biomarker-specific reagent for an EM marker, wherein the third, fourth, fifth and sixth detectable moieties (which may be the same or different from any of the first and second detectable moieties) are distinguishable from one another when labeling the same cell.

In another embodiment in which the FI Module 204 is programed to identify and categorize the objects of Feature Set 3 in separate digital images from the same tumor, the image analysis system may be programmed to execute a first FI Module 204 on a first digital image of a first mAHC-labeled section of a tumor and a second FI Module 204 on a second digital image of a second mAHC-labeled section of the tumor, wherein (a) the first FI Module 204 marks (a1) at least all of the cells in a tumor region that are CD8+, (a2) at least all of the cells in a tumor region that are EM+, (a3) at least all of the cells in a stroma region that are CD8+, and (a4) at least all cells in the stroma region that are PD-L1+/EM+; and (b) the second FI Module 204 marks (b1) at least all cells in a tumor region that are CD8+/PD-1+, and (b2) at least all cells in a tumor region that are CD8+/PD-L1+. The following feature metrics are then computed from the first digital image: (1) median distance of CD8+ cells from EM+ cells w/in 30 μm in the tumor region, and (2) median distance from CD8+ cells to PD-L1+/epithelial marker+ (EM+) cells within 10 μm in the stroma region; and the following feature metrics are computed from the second digital image: (3) mean number of CD8+/PD-1+ cells within 10 μm of CD8+/PD-L1+ cells, and (4) median distance from CD8+/PD-1+ cells to CD8+/PD-L1+ cells within 30 μm. In such an embodiment, the first mAHC-labeled section of the tumor may be labeled with a first detectable moiety via a biomarker specific reagent for CD8, a second detectable moiety via a biomarker-specific reagent for EM, and a third detectable moiety via a biomarker-specific reagent for PD-L1, wherein the first, second, and third detectable moieties are distinguishable from one another when labeling the same cell; and the second mAHC-labeled section of the tumor may be labeled with a fourth detectable moiety via a biomarker specific reagent for CD8, a fifth detectable moiety via a biomarker-specific reagent for PD-L1, a sixth detectable moiety via a biomarker-specific reagent for PD-1, and optionally a seventh detectable moiety via a biomarker-specific reagent for an EM marker, wherein the third, fourth, fifth and sixth detectable moieties (which may be the same or different from any of the first and second detectable moieties) are distinguishable from one another when labeling the same cell.

In another embodiment in which the FI Module 204 is programed to identify and categorize the objects of Feature Set 3 in separate digital images from the same tumor, the image analysis system may be programmed to execute a first FI Module 204 on a first digital image of a first mAHC-labeled section of a tumor, a second FI Module 204 on a second digital image of a second mAHC-labeled section of the tumor, and a third FI Module 204 on a third digital image of a third mAHC-labeled section of the tumor, wherein (a) the first FI Module 204 marks (a1) at least all cells in a tumor region that are CD8+, and (a2) at least all cells in a tumor region that are EM+; (b) the second FI Module 204 marks (b1) at least all cells in a stroma region that are CD8+ and (b2) at least all cells in a stroma region that are PD-L1+/EM+ cell; and (c) the third FI Module 204 marks (c1) at least all cells in a tumor region that are CD8+/PD-1+ and (c2) at least all cells in the tumor region that are CD8+/PD-L1+. The following feature metrics are then computed from the first digital image: (1) median distance of CD8+ cells from EM+ cells w/in 30 μm in the tumor region; the following feature metrics are then computed from the second digital image: (2) median distance from CD8+ cells to PD-L1+/epithelial marker+ (EM+) cells within 10 μm in the stroma region; and the following feature metrics are computed from the third digital image: (3) mean number of CD8+/PD-1+ cells within 10 μm of CD8+/PD-L1+ cells, and (4) median distance from CD8+/PD-1+ cells to CD8+/PD-L1+ cells within 30 μm. In such an embodiment, the first mAHC-labeled section of the tumor may be labeled with a first detectable moiety via a biomarker specific reagent for CD8 and a second detectable moiety via a biomarker-specific reagent for EM, wherein the first and second detectable moieties are distinguishable from one another when labeling the same cell; and the second mAHC-labeled section of the tumor may be labeled with a third detectable moiety via a biomarker specific reagent for CD8, a fourth detectable moiety via a biomarker-specific reagent for PD-L1, and a fifth detectable moiety via a biomarker-specific reagent for EM, wherein the third, fourth, and fifth detectable moieties (which may be the same as or different from any of the first and second detectable moieties) are distinguishable from one another when labeling the same cell; and the third mAHC-labeled section of the tumor may be labeled with a sixth detectable moiety via a biomarker-specific reagent for CD8, a seventh sixth detectable moiety via a biomarker-specific reagent for PD-1, an eight detectable moiety via a biomarker-specific reagent for PD-1, and optionally a ninth detectable moiety via a biomarker-specific reagent for an EM marker, wherein the sixth, seventh, eighth, and ninth detectable moieties (which may be the same or different from any of the first, second, third, fourth, and fifth detectable moieties) are distinguishable from one another when labeling the same cell.

In another embodiment, the FI Module 204 is programed to identify and categorize the objects of Feature Set 4 in a single digital image. In such an embodiment, the image analysis system is programmed to, for at least the cells in a tumor region, mark all cells that are CD8+ and all cells that are EM+. A density of CD8+/EM-cells in the tumor region is then computed. In such an embodiment, the mAHC-labeled section of the tumor may be labeled with a first detectable moiety via a biomarker specific reagent for CD8 and a second detectable moiety via a biomarker-specific reagent for EM, wherein the first and second detectable moieties are distinguishable from one another when labeling the same cell.

As used herein, detectable moieties are “distinguishable from one another when labeling the same cell” as long as the presence or absence of each moiety can be detected in the same cell.

### IV.B. ROI Module

The ROI Module 205 is used to generate the ROI or ROIs in the image from which the feature metrics are calculated.

In some embodiments, the ROI Module 205 generates a graphic user interface (GUI) through which a user manually annotates the ROI in the digital image. A trained expert (such as a pathologist) may use an user interaction device (such as a mouse, touchpad, stylus, touch-responsive display or the like) to delineate one or more morphological region(s) (such as a tumor area and/or an invasive front) on a representation of the digital image of the sample in the GUI. The area(s) delineated in the image may then be used as the ROI.

In other embodiments, the ROI Module 205 assists the user in annotating the ROI (termed, “semi-automated ROI annotation”). For example, the ROI Module 205 may generate a GUI containing the digital image. The user may use delineate one or more regions on the digital image, which the ROI Module 205 then automatically transforms into a complete ROI. For example, if the desired ROI is an PI, PO, and/or PR region, a user can delineate a tumor region and an invasive front, and the system automatically draws the PI, PO, and PR regions as defined by the user. In another embodiment, where the ROI is an EA or a SA, the user may delineate the tumor region and, optionally, the invasive front in the image, which is then registered to the biomarker-labeled image, and the system creates the relevant EA and SA ROIs by marking all cells within the pre-defined distance of an EM+ cell as being within the EA, and all cells beyond the pre-defined distance as being within the SA. As another example, the ROI Module 205 may apply a pattern recognition function that uses computer vision and machine learning to identify regions having similar morphological characteristics to delineated and/or auto-generated the ROIs. Thus, for example, a tumor region could be annotated in a semi-automated manner by a method comprising: (a) a user annotates the tumor region in an H&E image of the sample by outlining the tumor region; and (b) the ROI Module 205 applies a pattern recognition function to identify additional areas of the sample that have the morphological characteristics of the outlined area, wherein the overall tumor region includes the area annotated by the user and the areas automatically identified by the system. In another example, a PR, PI, and/or PO ROI could be annotated in a semi-automated manner by a method comprising: (a) a user annotates the tumor region in an H&E image of the sample by outlining the tumor region and invasive front; (b) the ROI Module 205 automatically defines the PR, PI, and/or PO region(s) encompassing all pixels within the defined distance of the annotated invasive front; and (c) the ROI Module 205 applies a pattern recognition function to identify additional areas of the sample that have the morphological characteristics of the PI, PO, and/or PR regions identified by step (b). Many other arrangements could be used as well. In cases in which ROI generation is semi-automated, the user may be given an option to modify the ROI annotated by the computer system, such as by expanding the ROI, annotating regions of the ROI or objects within the ROI to be excluded from analysis, etc.

In other embodiments, the ROI Module 205 may automatically suggest an ROI without any direct input from the user (termed an “automated ROI annotation”). For example, a previously-trained tissue segmentation function or other pattern recognition function may be applied to an unannotated image to identify the desired morphological region to use as an ROI. The user may be given an option to modify the ROI annotated by the computer system, such as by expanding the ROI, annotating regions of the ROI or objects within the ROI to be excluded from analysis, etc.

In yet other embodiments, ROI may be generated by using a registration function, whereby an ROI annotated in one section of a set of serial sections is automatically transferred to other sections of the set of serial sections. This functionality is especially useful when an H&E-stained serial section is provided along with the biomarker-labeled sections. In such an embodiment, the user may delineate, for example, the tumor region in the digital image of the H&E-stained section. The system then registers the ROI from the H&E image to the image of the biomarker-labeled serial section, matching the tissue structures from the H&E image to the corresponding tissue structures in the serial section. Exemplary registration methods can be found at, for example, WO2013/140070 and US 2016-0321809

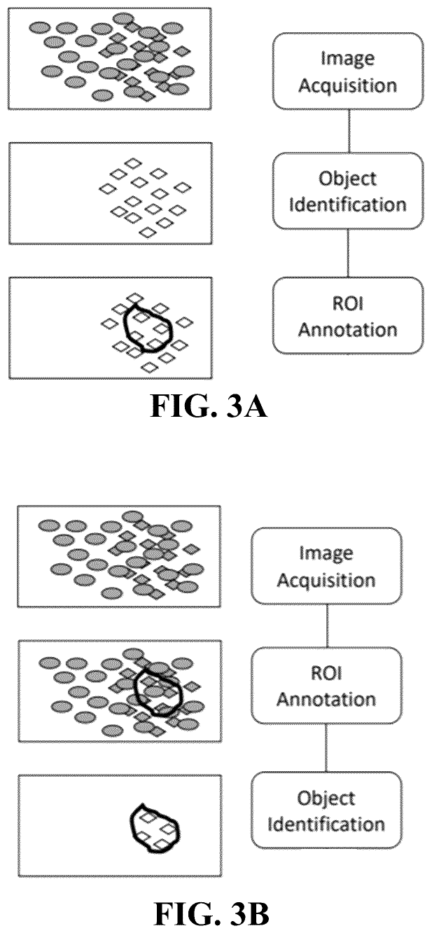

The FI Module 204 and the ROI Module 205 may be implemented in any order. For example, the FI Module 204 may be applied to the entire image first. The positions and feature vectors of the identified objects can then be stored and recalled later when the ROI Module 205 is implemented. In such an arrangement, a score can be generated by the Scoring Module 206 immediately upon generation of the ROI. Such a workflow is illustrated at FIG. 3A. As can be seen at FIG. 3A, an image is obtained having a mixture of different objects (illustrated by dark ovals and dark diamonds). After object identification task is implemented, all diamonds in the image are identified (illustrated by open diamonds). When the ROI is appended to the image (illustrated by the dashed line), only the diamonds located in the ROI region are included in the metric calculation for the ROI. Alternatively, the ROI Module 205 can be implemented first. Such a workflow is illustrated at FIG. 3B. As can be seen at FIG. 3B, an image is obtained having a mixture of different object (illustrated by dark ovals and dark diamonds). The ROI is appended to the image (illustrated by the dashed line), but no objects have been marked yet. The FI Module 204 may be implemented only on the ROI (which minimizes computation time), or it may still be implemented on the whole image (which would allow on-the-fly adjustments without re-running the FI Module 204). It is also possible to implement the FI Module 204 and ROI Module 205 simultaneously.

While these modules are depicted in FIG. 2 as standalone modules, it will be evident to persons having ordinary skill in the art that each module may instead be implemented as a number of sub-modules, and that in some embodiments any two or more modules can be combined into a single module. Furthermore, in some embodiments, image analysis system 200 may include additional modules (e.g., input devices, networking and communication modules, etc.) not depicted in FIG. 2 for brevity. Furthermore, in some embodiments, some of the blocks depicted in FIG. 2 may be disabled or omitted. As will be discussed in more detail below, the functionality of some or all modules of image analysis system 200 can be implemented in hardware, software, firmware, or as any combination thereof. Exemplary commercially-available software packages useful in implementing modules as disclosed herein include VENTANA VIRTUOSO; DEFINIENS TISSUE STUDIO, DEVELOPER XD, and IMAGE MINER; and VISOPHARM BIOTOPIX, ONCOTOPIX, and STEREOTOPIX software packages.

### IV.C. Scoring Module

After the FI Module 204 and ROI module 205 have been implemented, the Scoring Module 206 extracts relevant the feature metrics from the relevant ROI(s) and, optionally, computes the PRS by applying the feature metrics to a scoring function as described herein. The Scoring Module 206 is adapted to extract the relevant feature metrics from the annotated ROIs based on the feature vectors associated with the marked cells. This may be done after a final ROI has been selected or may be done continuously as the ROI is adjusted. For example, the FI Module 204 is applied to the whole image as described in FIG. 3A, generating a high level feature vector for each cell that includes biomarker status for all relevant cell types (regardless of ROI). The ROI module 205 and the Scoring Module 206 may then be applied simultaneously, in which case, as the ROI is adjusted by the ROI module 205, the Scoring Module adjusts the computed feature metrics (and optionally, a computed PRS) accordingly.

After the Scoring Module 206 has finished computing the feature metrics (and optionally the PRS), the final computed feature metrics and/or the PRS may be communicated to the output device 203. Where the scoring module 206 does not apply the scoring function to the feature metrics, the feature metrics are transmitted to the output device 203, which may either apply the scoring function to the feature metrics or may transmit feature metrics to an end user in a form that can be applied to a scoring function to generate a PRS (for example, by displaying or printing the raw feature metric data or exporting the raw feature metric data into a spreadsheet or other data analysis software program). Where the Scoring Module 206 applies the scoring function to the extracted feature metrics to compute the PRS, the PRS and, optionally, the raw feature metric data are exported to the output device 203. The PRS may be output as a numeric value or as a textual or graphical representation of the patients relative risk. For example, the output may be the numeric output of the scoring function when applied to the feature metrics (optionally along with appropriate cutoff values between risk buckets). As another example, the output may be a textual indication of the patients relative risk (i.e., by assigning the patient to a response bucket, such as “likely to respond” or “unlikely to respond”). As another example, the output may be a graphical representation of the patient's relative risk, such as a graph showing where the patient's score ranks among a population distribution. Many other outputs can be contemplated.

### IV.D. Exemplary Contemplated Embodiments

### IV.D.1. Feature Set 1

In an embodiment, the image analysis system 200 functions to compute the feature metrics of Feature Set 1 from a digital image of a single mAHC-labeled tissue sample. In such an embodiment, (a) the ROI Module 205 annotates a stroma region and a tumor region on the digital image; (b) the FI Module 204: (b1) marks objects corresponding to cells in the digital image; (b2) generates a feature vector indicating CD8, CD68, PD-L1, and optionally EM status for at least the cells in the stroma region; and (b3) generates a feature vector indicating CD8, PD-1, PD-L1, and optionally EM status for at least the cells in the tumor region; and (c) the Scoring Module 206 extracts the feature metrics of Feature Set 1 from their respective ROIs and, optionally, computes the PRS from the extracted feature metrics. In such an embodiment, the mAHC-labeled section of the tumor may be labeled with a first detectable moiety via a biomarker specific reagent for CD8, a second detectable moiety via a biomarker-specific reagent for CD68, a third detectable moiety via a biomarker-specific reagent for PD-L1, a fourth detectable moiety via a biomarker specific reagent for PD-1, and, optionally, a fifth detectable moiety via a biomarker-specific reagent for an EM, wherein the first, second, third, fourth, and fifth detectable moieties are distinguishable from one another when labeling the same cell.

In another embodiment, the image analysis system 200 functions to compute the feature metrics of Feature Set 1 in digital images of separate AHC-labeled tissue samples from the same tumor. In such an embodiment, the image analysis system 200 may be programmed to execute a set of functions on a first digital image of a first AHC-labeled section of a tumor and a second digital image of a second AHC-labeled section of the tumor, wherein (a) a first ROI Module 205 annotates a stroma region in the first digital image; (b) a first FI Module 204 marks objects corresponding to cells at least in the stroma region and generates a feature vector for each marked cell indicating CD8, CD68, PD-L1, and optionally EM status; (c) a first Scoring Module 206 computes a standard deviation of distance of CD8+ cells from CD68+/PDL1+ cells within 10 μm in the stroma region of the first digital image; (d) a second ROI Module 205 annotates a tumor region on the second digital image; (e) a second FI Module 204 marks objects corresponding to cells at least in the tumor region of the second digital image and generates a feature vector indicating CD8, PD-1, PD-L1, and optionally EM status for at least the cells in the tumor region; (f) a second Scoring Module 206 computes a mean distance of CD8+/PD-1+ cells from CD8+/PD-L1+ cells w/in 30 μm in the tumor region of the second digital image; and (g) an optional third Scoring Module 206 computes the PRS from the extracted feature metrics. In such an embodiment, the first digital image is obtained from a first mAHC-labeled section of the tumor labeled with a first detectable moiety via a biomarker specific reagent for CD8, a second detectable moiety via a biomarker-specific reagent for CD68, and a third detectable moiety via a biomarker-specific reagent for PD-L1, and optionally a fourth detectable moiety via a biomarker-specific reagent for EM (such as pan-cytokeratin), wherein the first, second, third, and optional fourth detectable moieties are distinguishable from one another when labeling the same cell; and the second digital image is obtained from a second mAHC-labeled section of the tumor labeled with a fifth detectable moiety via a biomarker specific reagent for CD8, a sixth detectable moiety via a biomarker-specific reagent for PD-1, a seventh detectable moiety via a biomarker-specific reagent for PD-L1, and optionally an eighth detectable moiety via a biomarker-specific reagent for EM (such as pancytokeratin), wherein the fifth, sixth, seventh, and optional eighth detectable moieties (which may be the same or different from the first, second, third, and optional fourth detectable moieties) are distinguishable from one another when labeling the same cell.

### IV.D.2. Feature Set 2

In an embodiment, the image analysis system 200 functions to compute the feature metrics of Feature Set 2 from a digital image of a single mAHC-labeled tissue sample. In such an embodiment, (a) the ROI Module 205 annotates a peritumor outside (PO region and a tumor region on the digital image; (b) the FI Module 204: (b1) marks objects corresponding to cells in the digital image; (b2) generates a feature vector indicating CD8, CD68, PD-L1, and optionally EM status for at least the cells in the PO region; and (b3) generates a feature vector indicating CD8 and optionally EM status for at least the cells in the tumor region; and (c) the Scoring Module 206 extracts the feature metrics of Feature Set 2 from their respective ROIs and, optionally, computes the PRS from the extracted feature metrics. In such an embodiment, the mAHC-labeled section of the tumor may be labeled with a first detectable moiety via a biomarker specific reagent for CD8, a second detectable moiety via a biomarker-specific reagent for CD68, a third detectable moiety via a biomarker-specific reagent for PD-L1, and, optionally, a fourth detectable moiety via a biomarker-specific reagent for an EM, wherein the first, second, third, and fourth detectable moieties are distinguishable from one another when labeling the same cell.

In another embodiment, the image analysis system 200 functions to compute the feature metrics of Feature Set 2 in digital images of separate AHC-labeled tissue samples from the same tumor. In such an embodiment, the image analysis system 200 may be programmed to execute a set of functions on a first digital image of a first AHC-labeled section of a tumor and a second digital image of a second AHC-labeled section of the tumor, wherein (a) a first ROI Module 205 annotates a peritumor outer (PO) region in the first digital image; (b) a first FI Module 204 marks objects corresponding to cells at least in the PO region and generates a feature vector for each marked cell indicating CD8, CD68, PD-L1, and optionally EM status; (c) a first Scoring Module 206 computes a standard deviation of distance of CD8+ cells from CD68+/PDL1+ cells within 10 μm in the PO region of the first digital image; (d) a second ROI Module 205 annotates a tumor region on the second digital image; (e) a second FI Module 204 marks objects corresponding to cells at least in the tumor region of the second digital image and generates a feature vector indicating CD8 and optionally EM status for at least the cells in the tumor region; (f) a second Scoring Module 206 computes a density of CD8+ cells in the tumor region of the second digital image; and (g) an optional third Scoring Module 206 computes the PRS from the extracted feature metrics. In such an embodiment, the first digital image is obtained from a first mAHC-labeled section of the tumor labeled with a first detectable moiety via a biomarker specific reagent for CD8, a second detectable moiety via a biomarker-specific reagent for CD68, and a third detectable moiety via a biomarker-specific reagent for PD-L1, and optionally a fourth detectable moiety via a biomarker-specific reagent for EM (such as pan-cytokeratin), wherein the first, second, third, and optional fourth detectable moieties are distinguishable from one another when labeling the same cell; and the second digital image is obtained from a second mAHC-labeled section of the tumor labeled with a fifth detectable moiety via a biomarker specific reagent for CD8, and an optional sixth detectable moiety via a biomarker-specific reagent for EM (such as pancytokeratin), wherein the fifth and optional sixth detectable moieties (which may be the same or different from the first, second, third, and optional fourth detectable moieties) are distinguishable from one another when labeling the same cell.

### IV.D.3. Feature Set 3

In an embodiment, the image analysis system 200 functions to compute the feature metrics of Feature Set 3 from a digital image of a single mAHC-labeled tissue sample. In such an embodiment, (a) the ROI Module 205 annotates a tumor region and a stroma region on the digital image; (b) the FI Module 204: (b1) marks objects corresponding to cells in the digital image, generates a feature vector indicating CD8, PD-1, PD-L1, and EM status for at least the cells in the tumor region, and generates a feature vector indicating CD8, PD-L1, and EM status for at least the cells in the stroma region; and (c) the Scoring Module 206 extracts the feature metrics of Feature Set 3 from their respective ROIs and, optionally, computes the PRS from the extracted feature metrics. In such an embodiment, the mAHC-labeled section of the tumor may be labeled with a first detectable moiety via a biomarker specific reagent for CD8, a second detectable moiety via a biomarker-specific reagent for PD-1, a third detectable moiety via a biomarker-specific reagent for PD-L1, and, a fourth detectable moiety via a biomarker-specific reagent for an EM (such as pancytokeratin), wherein the first, second, third, and fourth detectable moieties are distinguishable from one another when labeling the same cell.

In another embodiment, the image analysis system 200 functions to compute the feature metrics of Feature Set 3 in digital images of separate AHC-labeled tissue samples from the same tumor. In such an embodiment, the image analysis system 200 may be programmed to execute a set of functions on a first digital image of a first AHC-labeled section of a tumor and a second digital image of a second AHC-labeled section of the tumor, wherein (a) a first ROI Module 205 annotates a tumor region in the first digital image; (b) a first FI Module 204 marks objects corresponding to cells at least in the tumor region and generates a feature vector for each marked cell indicating CD8, PD-1, PD-L1, and EM status; (c) a first Scoring Module 206 computes (c1) a median distance from CD8+ cells to EM+ cells within 30 μm, (c2) a mean number of CD8+/PD-1+ cells within 10 μm of CD8+/PD-L1+ cells, and (c3) a mean distance from CD8+/PD-1+ cells to CD8+/PD-L1+ cells within 30 μm from the tumor region of the first digital image; (d) a second ROI Module 205 annotates a stroma region on the second digital image; (e) a second FI Module 204 marks objects corresponding to cells at least in the stroma region of the second digital image and generates a feature vector indicating CD8, PD-L1, and EM status for at least the cells in the stroma region; (f) a second Scoring Module 206 computes mean distance from CD8+ cells to PD-L1+/EM+ cells within 10 μm in the stroma region in the tumor region of the second digital image; and (g) an optional third Scoring Module 206 computes the PRS from the extracted feature metrics. In such an embodiment, the first digital image is obtained from a first mAHC-labeled section of the tumor labeled with a first detectable moiety via a biomarker specific reagent for CD8, a second detectable moiety via a biomarker-specific reagent for PD-1, and a third detectable moiety via a biomarker-specific reagent for PD-L1, and a fourth detectable moiety via a biomarker-specific reagent for EM (such as pan-cytokeratin), wherein the first, second, third, and fourth detectable moieties are distinguishable from one another when labeling the same cell; and the second digital image is obtained from a second mAHC-labeled section of the tumor labeled with a fifth detectable moiety via a biomarker specific reagent for CD8, and a sixth detectable moiety via a biomarker-specific reagent for PD-L1, and a seventh detectable moiety via a biomarker-specific reagent for EM (such as pancytokeratin), wherein the fifth, sixth, and seventh detectable moieties (which may be the same or different from the first, second, third, and fourth detectable moieties) are distinguishable from one another when labeling the same cell.

In another embodiment, the image analysis system 200 functions to compute the feature metrics of Feature Set 3 in digital images of separate AHC-labeled tissue samples from the same tumor. In such an embodiment, the image analysis system 200 may be programmed to execute a set of functions on a first digital image of a first AHC-labeled section of a tumor and a second digital image of a second AHC-labeled section of the tumor, wherein (a) a first ROI Module 205 annotates a tumor region and a stroma region in the first digital image; (b) a first FI Module 204 (b1) marks objects corresponding to cells in the digital image, (b2) generates a feature vector for at least each marked cell in the tumor region indicating CD8 and EM status, and (b3) generates a feature vector for at least each marked cell in the stromal region indicating CD8, PD-L1, and EM status; (c) a first Scoring Module 206 computes (c1) median distance of CD8+ cells from EM+ cells w/in 30 μm in the tumor region of the first digital image and (c2) median distance from CD8+ cells to PD-L1+/epithelial marker+ (EM+) cells within 10 μm in the stroma region of the first digital image; (d) a second ROI Module 205 annotates a tumor region on the second digital image; (e) a second FI Module 204 marks objects corresponding to cells at least in the tumor region of the second digital image and generates a feature vector indicating CD8, PD-1, PD-L1, and optionally EM status for at least the cells in the tumor region; (f) a second Scoring Module 206 computes (f1) a mean number of CD8+/PD-1+ cells within 10 μm of CD8+/PD-L1+ cells in the tumor region of the second digital image and (f2) a distance from CD8+/PD-1+ cells to CD8+/PD-L1+ cells within 30 μm in the tumor region of the second digital image; and (g) an optional third Scoring Module 206 computes the PRS from the extracted feature metrics. In such an embodiment, the first mAHC-labeled section of the tumor may be labeled with a first detectable moiety via a biomarker specific reagent for CD8, a second detectable moiety via a biomarker-specific reagent for EM, and a third detectable moiety via a biomarker-specific reagent for PD-L1, wherein the first, second, and third detectable moieties are distinguishable from one another when labeling the same cell; and the second mAHC-labeled section of the tumor may be labeled with a fourth detectable moiety via a biomarker specific reagent for CD8, a fifth detectable moiety via a biomarker-specific reagent for PD-L1, a sixth detectable moiety via a biomarker-specific reagent for PD-1, and optionally a seventh detectable moiety via a biomarker-specific reagent for an EM marker, wherein the third, fourth, fifth and sixth detectable moieties (which may be the same or different from any of the first and second detectable moieties) are distinguishable from one another when labeling the same cell.

In another embodiment, the image analysis system 200 functions to compute the feature metrics of Feature Set 3 in digital images of separate AHC-labeled tissue samples from the same tumor. In such an embodiment, the image analysis system 200 may be programmed to execute a set of functions on a first digital image of a first AHC-labeled section of a tumor, a second digital image of a second AHC-labeled section of the tumor, and a third digital image of a third AHC-labeled section of a tumor, wherein: (a) a first ROI Module 205 annotates a tumor region in the first digital image; (b) a first FI Module 204 marks objects corresponding to cells in at least the tumor region of the first digital image and generates a feature vector for at least each marked cell in the tumor region indicating CD8 and EM status; (c) a first Scoring Module 206 computes a median distance of CD8+ cells from EM+ cells w/in 30 μm in the tumor region of the first digital image; (d) a second ROI Module 205 annotates a stroma region on the second digital image; (e) a second FI Module 204 marks objects corresponding to cells at least in the stroma region of the second digital image and generates a feature vector indicating CD8, PD-L1, and EM status for at least the cells in the stroma region; (f) a second Scoring Module 206 computes median distance from CD8+ cells to PD-L1+/EM+ cells within 10 μm in the stroma region of the second digital image; (g) a third ROI Module 205 annotates a tumor region on the third digital image; (h) a third FI Module 204 marks objects corresponding to cells at least in the tumor region of the third digital image and generates a feature vector indicating CD8, PD-1, PD-L1, and optionally EM status for at least the cells in the tumor region; (i) a third Scoring Module 206 computes (i1) a mean number of CD8+/PD-1+ cells within 10 μm of CD8+/PD-L1+ cells and (i2) a median distance from CD8+/PD-1+ cells to CD8+/PD-L1+ cells within 30 μm; and (j) an optional fourth Scoring Module 206 computes the PRS from the extracted feature metrics. In such an embodiment, the first mAHC-labeled section of the tumor may be labeled with a first detectable moiety via a biomarker specific reagent for CD8 and a second detectable moiety via a biomarker-specific reagent for EM, wherein the first and second detectable moieties are distinguishable from one another when labeling the same cell; and the second mAHC-labeled section of the tumor may be labeled with a third detectable moiety via a biomarker specific reagent for CD8, a fourth detectable moiety via a biomarker-specific reagent for PD-L1, and a fifth detectable moiety via a biomarker-specific reagent for EM, wherein the third, fourth, and fifth detectable moieties (which may be the same as or different from any of the first and second detectable moieties) are distinguishable from one another when labeling the same cell; and the third mAHC-labeled section of the tumor may be labeled with a sixth detectable moiety via a biomarker-specific reagent for CD8, a seventh sixth detectable moiety via a biomarker-specific reagent for PD-1, an eight detectable moiety via a biomarker-specific reagent for PD-1, and optionally a ninth detectable moiety via a biomarker-specific reagent for an EM marker, wherein the sixth, seventh, eighth, and ninth detectable moieties (which may be the same or different from any of the first, second, third, fourth, and fifth detectable moieties) are distinguishable from one another when labeling the same cell.

### IV.D.4. Feature Set 4

In an embodiment, the image analysis system 200 functions to compute the feature metrics of Feature Set 4 from a digital image of a single AHC-labeled tissue sample. In such an embodiment, (a) the ROI Module 205 annotates a tumor region on the digital image; (b) the FI Module 204: (b1) marks objects corresponding to cells in the digital image and generates a feature vector indicating CD8 and optionally EM status for at least the cells in the tumor region; and (c) the Scoring Module 206 extracts the feature metrics of Feature Set 4 from their respective ROI and, optionally, computes the PRS from the extracted feature metrics. In such an embodiment, the AHC-labeled section of the tumor may be labeled with a first detectable moiety via a biomarker specific reagent for CD8, and, optionally, a second detectable moiety via a biomarker-specific reagent for an EM (such as pancytokeratin), wherein the first and second detectable moieties are distinguishable from one another when labeling the same cell.

### IV.D.5. Feature Set 5

In an embodiment, the image analysis system 200 functions to compute the feature metrics of Feature Set 5 from a digital image of a single AHC-labeled tissue sample. In such an embodiment, (a) the ROI Module 205 annotates a tumor region on the digital image; (b) the FI Module 204: (b1) marks objects corresponding to cells in the digital image and generates a feature vector indicating PD-1 and PD-L1 status for at least the cells in the tumor region; and (c) the Scoring Module 206 extracts the feature metrics of Feature Set 5 from their respective ROI and, optionally, computes the PRS from the extracted feature metrics. In such an embodiment, the AHC-labeled section of the tumor may be labeled with a first detectable moiety via a biomarker specific reagent for PD-1, and a second detectable moiety via a biomarker-specific reagent for PD-L1, wherein the first and second detectable moieties are distinguishable from one another when labeling the same cell. In some embodiments, the detectable moieties are brightfield dyes or fluorescent dyes.

### IV.E. Sample Analysis Systems

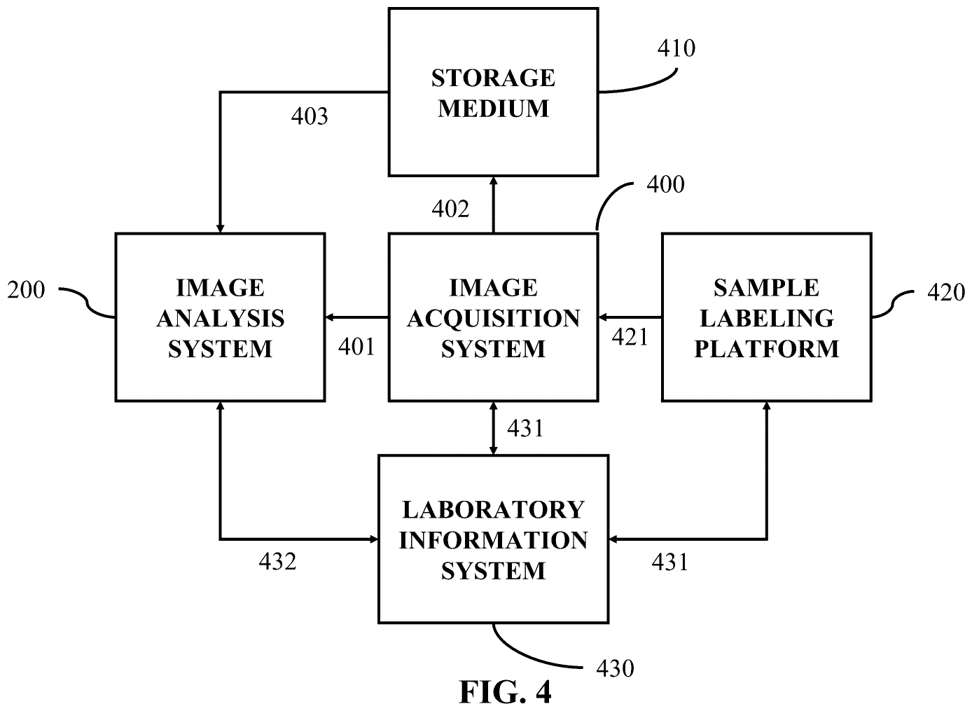

In some embodiments, image analysis system 200 may be implemented in combination with one or more additional systems to form a sample analysis system. An exemplary sample analysis system is illustrated at FIG. 4.

For example, the image analysis system 200 may work in combination with an image acquisition system 400. Image acquisition system 400 generates digital images of AHC-stained samples and provide those images to image analysis system 200 for analysis and presentation to the user. Image acquisition system 400 may include a scanning platform, such as a slide scanner that can scan slides containing AHC-labeled samples at 20×, 40×, or other magnifications to produce high resolution whole-slide digital images. At a basic level, the typical slide scanner includes at least: (1) a microscope with lens objectives, (2) a light source (such as halogen, light emitting diode, white light, and/or multispectral light sources, depending on the dye), (3) robotics to move glass slides around (or to move the optics around the slide), (4) one or more digital cameras for image capture, (5) a computer and associated software to control the robotics and to manipulate, manage, and view digital slides. Digital data at a number of different X-Y locations (and in some cases, at multiple Z planes) on the slide are captured by the camera's charge-coupled device (CCD), and the images are joined together to form a composite image of the entire scanned surface. Common methods to accomplish this include:
(1) Tile based scanning, in which the slide stage or the optics are moved in very small increments to capture square image frames, which overlap adjacent squares to a slight degree. The captured squares are then automatically matched to one another to build the composite image; and
(2) Line-based scanning, in which the slide stage moves in a single axis during acquisition to capture a number of composite image “strips.”

The image strips can then be matched with one another to form the larger composite image. A detailed overview of various scanners (both fluorescent and brightfield) can be found at Farahani et al., Whole slide imaging in pathology: advantages, limitations, and emerging perspectives, Pathology and Laboratory Medicine Int'l, Vol. 7, p. 23-33 (June 2015), the content of which is incorporated by reference in its entirety. Examples of commercially available slide scanners include: 3DHistech PANNORAMIC SCAN II; DigiPath PATHSCOPE; Hamamatsu NANOZOOMER RS, HT, and XR; Huron TISSUESCOPE 4000, 4000XT, and HS; Leica SCANSCOPE AT, AT2, CS, FL, and SCN400; Mikroscan D2; Olympus VS120-SL; Omnyx VL4, and VL120; PerkinElmer LAMINA; Philips ULTRA-FAST SCANNER; Sakura Finetek VISIONTEK; Unic PRECICE 500, and PRECICE 600x; VENTANA ISCAN COREO and ISCAN HT; and Zeiss AXIO SCAN.Z1. Other exemplary systems and features can be found in, for example, WO2011-049608) or in U.S. Patent Application No. 61/533,114, filed on Sep. 9, 2011, entitled IMAGING SYSTEMS, CASSETTES, AND METHODS OF USING THE SAME the content of which is incorporated by reference in its entirety.

In some embodiments, the images generated by the Image Acquisition System 400 may be communicatively coupled to the image analysis system 200 such that the image can be transferred directly 401, for example, via one or more local-area networks and/or wide-area networks or via a shared memory. In some embodiments, image acquisition system 400 may not be communicatively coupled to image analysis system 200, in which case the images may be stored on a storage medium 410 (such as a non-volatile storage medium of any type (e.g., a flash drive) or on a server or database accessible by image analysis system)). In such a case, the image analysis system 200 may download the image 403.

The sample analysis system may also include one or more sample labeling platforms 420, such as an automated AHC platform and/or an automated H&E staining platform. Stained samples generated by the sample labeling platform are the transferred for imaging 421 to the image acquisition system 400. The resulting digital images are then transferred 401-403 to the image analysis system 200 for evaluation.

Automated AHC platforms typically include at least: reservoirs of the various reagents used in the labeling protocols, a reagent dispense unit in fluid communication with the reservoir(s) for dispensing reagent to onto a slide, a waste removal system for removing used reagents and other waste from the slide, and a control system that coordinates the actions of the reagent dispense unit and waste removal system. In addition to performing labeling steps, many automated slide stainers can also perform steps ancillary to labeling (or are compatible with separate systems that perform such ancillary steps), including: slide baking (for adhering the sample to the slide), dewaxing (also referred to as deparaffinization), antigen retrieval, counterstaining, dehydration and clearing, and coverslipping. Prichard, Overview of Automated Immunohistochemistry, Arch Pathol Lab Med., Vol. 138, pp. 1578-1582 (2014), incorporated herein by reference in its entirety, describes several specific examples of automated IHC/ISH slide stainers and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and Lab Vision Autostainer (Thermo Scientific) automated slide stainers. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published patents application Nos. 20030211630 and 20040052685, each of which is incorporated herein by reference in its entirety. Commercially-available staining units typically operate on one of the following principles: (1) open individual slide staining, in which slides are positioned horizontally and reagents are dispensed as a puddle on the surface of the slide containing a tissue sample (such as implemented on the DAKO AUTOSTAINER Link 48 (Agilent Technologies) and intelliPATH (Biocare Medical) stainers); (2) liquid overlay technology, in which reagents are either covered with or dispensed through an inert fluid layer deposited over the sample (such as implemented on BENCHMARK and DISCOVERY stainers); (3) capillary gap staining, in which the slide surface is placed in proximity to another surface (which may be another slide or a coverplate) to create a narrow gap, through which capillary forces draw up and keep liquid reagents in contact with the samples (such as the staining principles used by DAKO TECHMATE, Leica BOND, and DAKO OMNIS stainers). Some iterations of capillary gap staining do not mix the fluids in the gap (such as on the DAKO TECHMATE and the Leica BOND). In variations of capillary gap staining termed dynamic gap staining, capillary forces are used to apply sample to the slide, and then the parallel surfaces are translated relative to one another to agitate the reagents during incubation to effect reagent mixing (such as the staining principles implemented on DAKO OMNIS slide stainers (Agilent)). In translating gap staining, a translatable head is positioned over the slide. A lower surface of the head is spaced apart from the slide by a first gap sufficiently small to allow a meniscus of liquid to form from liquid on the slide during translation of the slide. A mixing extension having a lateral dimension less than the width of a slide extends from the lower surface of the translatable head to define a second gap smaller than the first gap between the mixing extension and the slide. During translation of the head, the lateral dimension of the mixing extension is sufficient to generate lateral movement in the liquid on the slide in a direction generally extending from the second gap to the first gap. See WO 2011-139978 A1. It has recently been proposed to use inkjet technology to deposit reagents on slides. See WO 2016-170008 A1. This list of staining technologies is not intended to be comprehensive, and any fully or semi-automated system for performing biomarker staining may be useful.

Automated H&E staining platforms typically operate on one of two staining principles: batch staining (also referred to as “dip 'n dunk”) or individual slide staining. Batch stainers generally use vats or baths of reagents in which many slides are immersed at the same time. Individual slide stainers, on the other hand, apply reagent directly to each slide, and no two slides share the same aliquot of reagent. Examples of commercially available H&E stainers include the VENTANA SYMPHONY (individual slide stainer) and VENTANA HE 600 (individual slide stainer) series H&E stainers from Roche; the DAKO COVERSTAINER (batch stainer) from Agilent Technologies; the LEICA ST4020 SMALL LINEAR STAINER (batch stainer), LEICA ST5020 MULTISTAINER (batch stainer), and the LEICA ST5010 AUTOSTAINER XL series (batch stainer) H&E stainers from Leica Biosystems Nussloch GmbH. H&E staining platforms are typically used in workflows in which a morphologically-stained serial section of the biomarker-labeled section(s) is desired.

The sample analysis system may further include a laboratory information system (LIS) 420. LIS 430 typically performs one or more functions selected from: recording and tracking processes performed on samples and images derived from the samples, instructing different components of the sample analysis system to perform specific processes on the samples, slides, and/or images, and track information about specific reagents applied to samples and or slides (such as lot numbers, expiration dates, volumes dispensed, etc.). LIS 430 usually comprises at least a database containing information about samples; labels associated with samples, slides, and/or image files (such as barcodes (including 1-dimensional barcodes and 2-dimensional barcodes), radio frequency identification (RFID) tags, alpha-numeric codes affixed to the sample, and the like); and a communication device that reads the label on the sample or slide and/or communicates information about the slide between the LIS 430 and the other components of the Sample analysis system. Thus, for example, a communication device could be placed at each of a sample processing station (not pictured), sample labeling platform(s) 420, and image acquisition system 400. When the sample is initially processed into sections, information about the sample (such as patient ID, sample type, processes to be performed on the section(s)) may be entered into the communication device, and a label is created for each section generated from the sample. At each subsequent station, the label is entered into the communication device (such as by scanning a barcode or RFID tag or by manually entering the alpha-numeric code), and the station electronically communicates with the LIS 430 to, for example, instruct the station or station operator to perform a specific process on the section and/or to record processes being performed on the section 431. The image acquisition system 400 may also encode each image with a computer-readable label or code that correlates back to the section or sample from which the image is derived, such that when the image is sent to the image analysis system 200, image processing steps to be performed may be sent from the LIS to the image analysis system and/or image processing steps performed on the image by image analysis system are recorded by database of LIS 432. Additionally, the LIS 430 may function as the output device for the image analysis system 200, wherein the extracted feature metrics and/or computed PRS are transmitted to and stored on the LIS 432. Commercially available LIS systems useful in the present methods and systems include, for example, VENTANA VANTAGE WORKFLOW system (Roche).

### V. Examples

### Example 1: Multiplex Fluorescent Immunohistochemical Assays

Three multiplex fluorescent IHC (mfIHC) panels were designed as described in Table 9:

TABLE 9

| | | |
|---|---|---|
| Panel A | Panel B | Panel C |

| Biomarker | Label | Biomarker | Label | Biomarker | Label |
|---|---|---|---|---|---|
| CD3 | Cy5 | CD8 | Cy5 | MHCI | Cy5 |
| CD68 | R610 | PanCK | R610 | CD14 | R610 |
| CD8 | R6G | PD-1 | R6G | TGFBR2 | R6G |
| PD-L1 | FAM | PD-L1 | FAM | B2M | FAM |
| PanCK | DCC | Lag3 | DCC | PanCK | DCC |

The following antibody clones were used: CD3 (SP162), CD8 (SP239), CD68 (SP251), PD-L1 (SP263), pan-cytokeratin (panCK) (AE1/AE3/PCK26 cocktail), PD1 (NAT105), LAG3 (E17B4), MHC-I (EP1395Y), β2-microglobulin (B2M) (ERP21752-214), CD14 (EPR3653), and TGF-β receptor 2 (TGFBR2) (MBS2400063).

The mfIHC panels were applied to 4 μM tissue sections of formalin fixed paraffin embedded (FFPE) tissue blocks from a cohort of dMMR/MSI-H mCRC patients treated with pembrolizumab monotherapy. The detailed description of epitope retrieval from FFPE tissue sections, antibody titration, incubation and image acquisition were previously described (Zhang II). In brief, for each target, the corresponding 1° antibody (1° Ab) was incubated on the slide, followed by a horseradish peroxidase (HRP) conjugated 2° Ab goat anti-mouse-HRP (#760-7060) for PD1, LAG3, and PanCK; and goat anti-rabbit-HRP (#760-7058) for CD8, CD68, CD14, MHCI, TGFBR2, B2M, CD3 and PDL1; the target was then detected with a tyramide-conjugated fluorophore (TSA-FL): DISCOVERY Red 610 kit (#760-245, Roche) (fluorophore having an excitation wavelength of 580 nm and an emission wavelength of 625 nm (“R610”)), DISCOVERY Rhodamine 6G kit (#760-244, Roche) (fluorophore having an excitation wavelength of 546 nm and an emission wavelength of 572 nm (“R6G”)), DISCOVERY FAM kit (#760-243, Roche) (fluorophore having an excitation wavelength of 490 nm and an emission wavelength of 520 nm (“FAM”)), DISCOVERY Cy5 kit (#760-238, Roche) (fluorophore having an excitation wavelength of 650 nm and an emission wavelength of 670 nm (“Cy5”)), DISCOVERY DCC kit (#760-240, Roche) (fluorophore having an excitation wavelength of 436 nm and an emission wavelength of 480 nm (“DCC”)). The next target detection followed the same scheme, and so on. To prevent potential cross-reaction of same species 1° antibodies, a heating step was introduced to deactivate the 1° Ab & 2° Ab complex before detecting the next target. Slides were then counterstained with 4′,6-diamidino-2-phenylindole (DAPI) (Cat #760-4196, Roche). Slides were cover slipped using micro cover glass, 24×50 mm no. 1.5 (VWR, Cat #: 48393241) and a PROLONG DIAMOND Antifade Mountant with DAPI (ThermoFisher Scientific, Cat #: P36962). Once the slides are stained they are cover slipped. The cover slip is mounted with Diamond prolong mounting media and reaches its optimal optical properties after 24 hours. Then the tissue is outlined using a sharpie on the glass slide by hand under a dissection scope and cleaned to remove dust and debris.

Additionally, a serial section of each sample labeled with the mfIHC assay was stained with hematoxylin and eosin (H&E) on a VENTANA HE 600 slide stainer.

### Example 2: Image Scanning and Image Analysis

The fluorescent image acquisition was performed on a ZEISS AXIO SCAN.Z1 slide scanner (Oberkochen, Germany). The slides were placed into the slide scanner where they were imaged based on panel of markers. The filters were custom narrow banded filters designed for our 6 markers per each channel. The exposure time for each marker was maintained throughout the experiment to ensure the intensity and populations as compared to the DAB during validation. The image was evaluated for quality and rescanned (if necessary) until a sharp clean image was acquired.

Image analysis was performed on HALO image analysis platform (Indica Labs). Using an H&E slide stained from the same case, a pathologist annotated the ROI. Within the HALO software, trained classifiers were applied to distinguish epithelial tumor from tumor stroma, to remove artifacts, to detect individual nuclei, and to expand a cytoplasmic radius range from the detected nucleus boundary. Each cell was defined as positive or negative for each marker based on intensity thresholding. The analysis setting was executed on the annotated region, and quality checked by a second qualified user.

From the HALO platform, a csv file with the raw data for each cell was downloaded. A python script was developed and validated to read the raw data and populate the features, including numbers of cells, density, intensity, ratios/fractions, percentages, and spatial relationships unique to each panel. The script was automated to report a batch of readouts from a batch of raw data outputs for the regions of interest from each case.

### Example 3: Model Generation

The features generated using the panel specific readout scripts were jointly used to develop a model to stratify subjects into low and high risk groups. The model development procedure may be broken down into 3 steps: Feature Pre-Processing, Feature Selection, Model Fitting and Case Stratification.

### Example 3A: Pre-Processing

Feature Pre-Processing was performed to remove data artifacts which may negatively impact the quality of feature selection and model fitting results in later steps.

First, features with missing readout data for >50% of cases were removed to reduce model bias where the available data may not have accurately described the underlying feature distribution. For features where ≤50% of data was missing across cases, imputation was performed.

Second, features that provide redundant information are removed based on assessment of pair-wise correlation. Features where Mean and Median cell intensity was computed for identical phenotype were assessed, and in cases where correlation exceeded 99% the Mean feature was removed from consideration. This step removed feature which portray identical information, to reduce risk of overfitting in downstream feature selection and model fitting.

Third, features whose value is dependent on tissue size were removed or re-formatted to be normalized by tissue size. In the feature set under consideration, this including all features which are counts of the number of cells. Because the density feature is a normalized representation of the number of cells, the features which consisted of number of cells were removed from consideration in downstream feature selection and model fitting.

### Example 3B: Feature Selection

Feature selection was performed using a Cox regression model with LASSO regularization. The choice of regularization weight was determined by varying the regularization parameter, and performing cross validation at each weight. The weight which minimized the partial likelihood deviance was chosen as a candidate. Due to the small size of the training dataset, defined as number of observations (cases) per feature, and the randomness of data partitioning during cross-validation, several iterations (e.g. 1000) of cross validation were performed for a given regularization weight, and the average candidate weight among iterations was chosen as the final weight. Given the final weight, a final regularized Cox regression model was trained. The features with non-zero coefficients from the final model were then selected as features.

Separate models were trained using both Progression Free Survival and Overall Survival as a response variable. Additionally, training was performed with variables from individual panels and all panels considered for feature selection.

### Example 3C: Model Fitting

A Cox Proportional Hazard model was fit using the set of features chosen during Feature Selection. The coefficients of the fitted hazard model were used to compute a risk score for each subject. The median risk score was used as a cutoff to divide the case population into low-risk and high-risk groups, with predicted responders in the low risk group and predicted non-responders in the high risk group. Four separate models were developed.

### Example 3D: Model 1

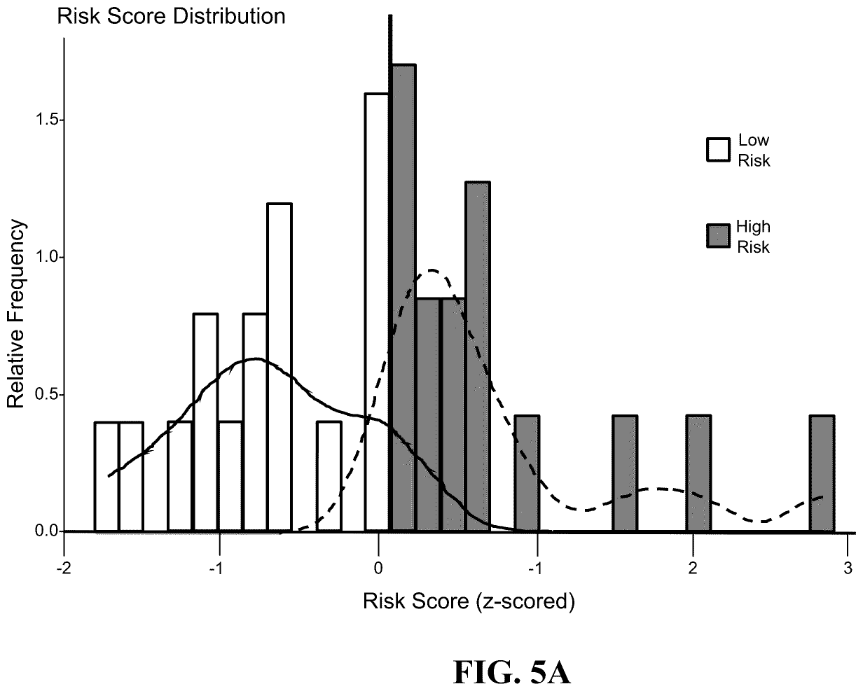

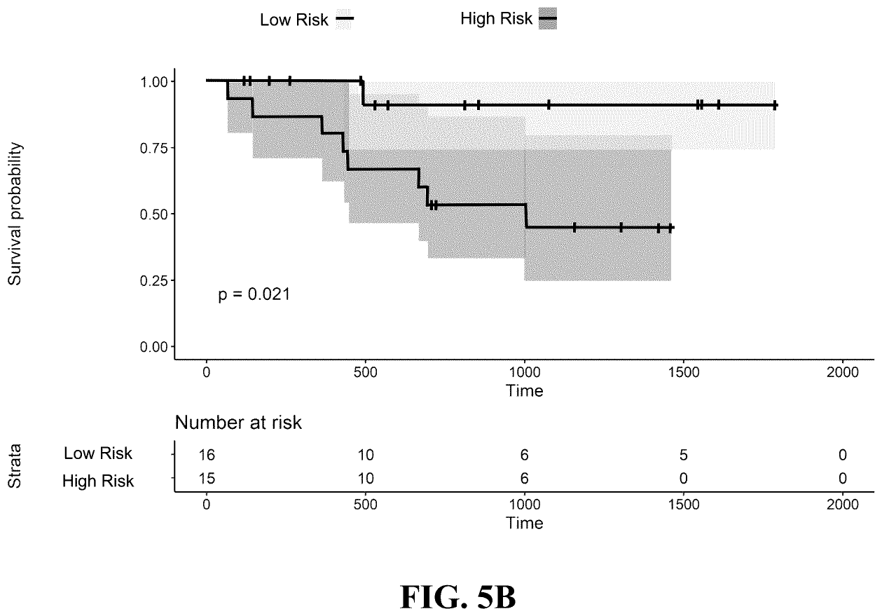

Model 1 was fit using one feature metric from mfIHC panel A and one feature from mfIHC panel B from Table 9 using overall survival as the relevant outcome. Model 1 details are summarized below at Table 10. The risk score distribution is illustrated at FIG. 5A (vertical black line illustrates stratification cutoff). Kaplan-Meier curve and the associated patient stratification chart are illustrated at FIG. 5B.

TABLE 10
MODEL 1

| | | | | mfIHC |
|---|---|---|---|---|
| Feature Metric | ROI | HR | P-value | panel |
| SDD of CD8+ from | SR | 0.1316 | 0.02615 | 1 |
| CD68+/PDL1+ w/in 10 μm | | | | |
| MnD of CD8+/PD1+ from | TR | 1.588 | 0.00862 | 2 |
| CD8+/PDL1+ within 30 μm | | | | |

| | |
|---|---|
| Model Evaluation | Likelihood ratio test = |
| | 10.09 on 2 df, p = 0.006 |

HR: hazard ratio;
MnD: mean distance;
SDD: standard deviation of distance;
SR: stroma region;
TR: tumor region

### Example 3E: Model 2

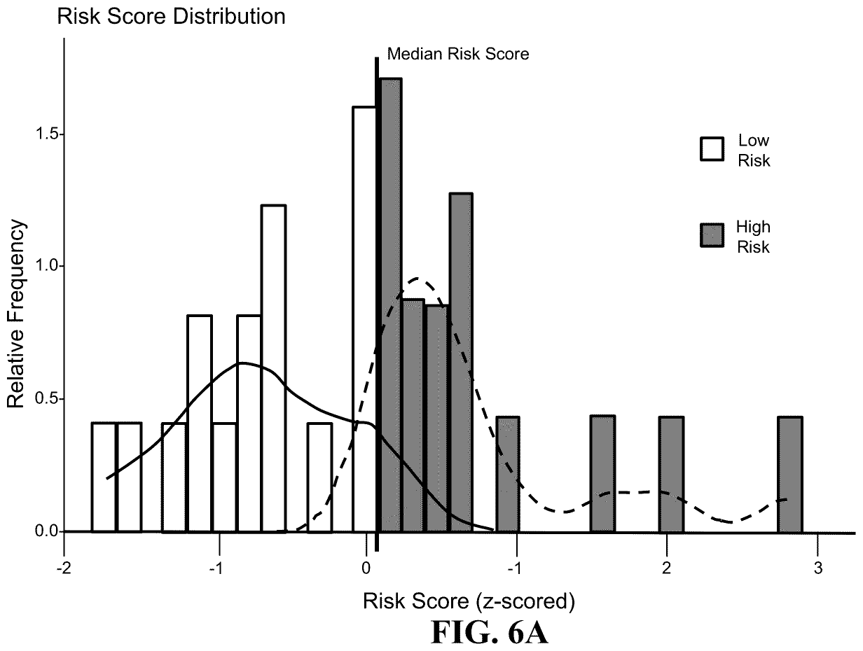

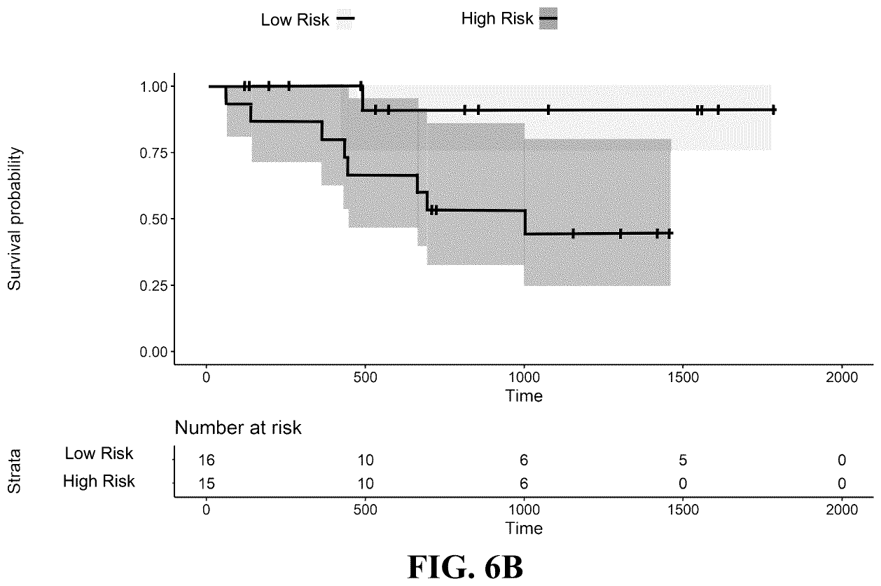

Model 2 was fit using one feature metric from mfIHC panel A and one feature from mfIHC panel B from Table 9 using overall survival as the relevant outcome. Model 2 details are summarized below at Table 11. The risk score distribution is illustrated at FIG. 6A (vertical black line illustrates stratification cutoff). A Kaplan-Meier curve and the associated patient stratification chart are illustrated at FIG. 6B.

TABLE 11
MODEL 2

| | | | | mfIHC |
|---|---|---|---|---|
| Feature Metric | ROI | HR | P-value | panel |
| SDD of CD8+ from | PO | 0.1299 | 0.0827 | 1 |
| CD68+/PDL1+ w/in 10 μm | | | | |
| Density of CD8+/PanCK− | TR | 0.4054 | 0.0669 | 2 |
| cells (100 cells/mm²) | | | | |

| | |
|---|---|
| Model Evaluation | Likelihood ratio test = |
| | 10.09 on 2 df, p = 0.006 |

HR: hazard ratio;
MnD: mean distance;
SDD: standard deviation of distance;
SR: stroma region;
TR: tumor region

### Example 3F: Model 3

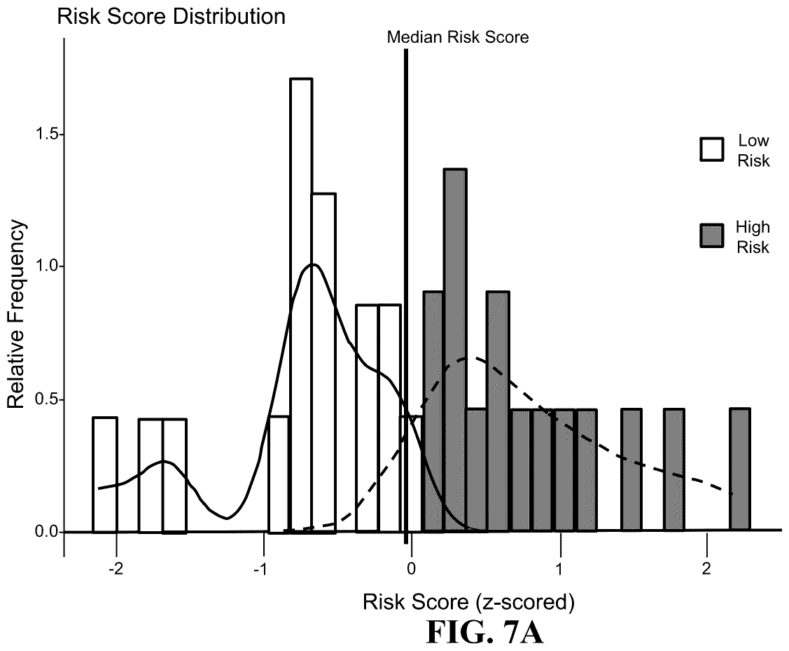

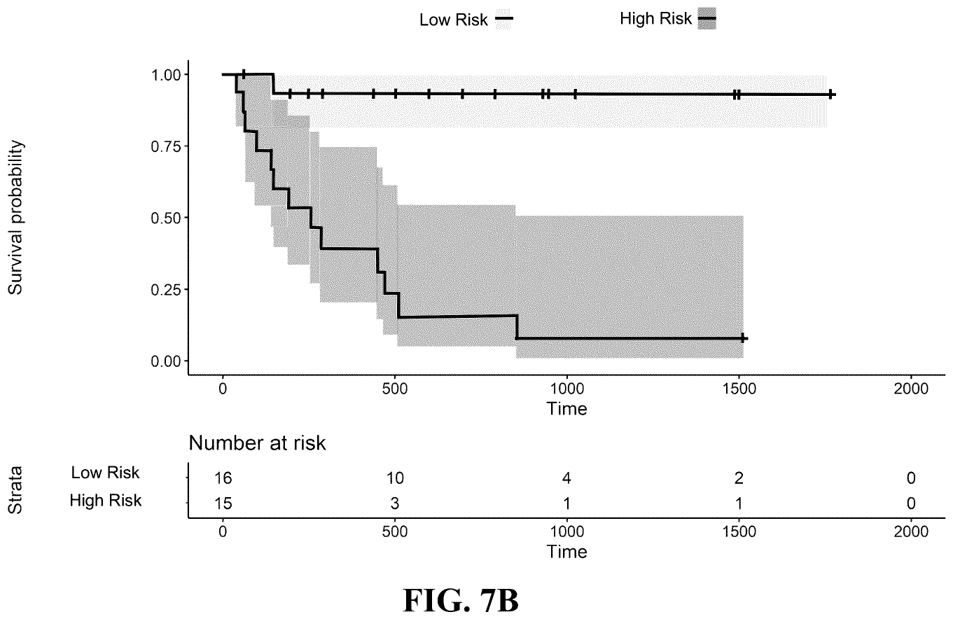

Model 3 was fit using two feature metrics from mfIHC panel A and two feature metrics from mfIHC panel B from Table 9 using progression-free survival as the relevant outcome. Model 3 details are summarized below at Table 12. The risk score distribution is illustrated at FIG. 7A (vertical black line illustrates stratification cutoff). A Kaplan-Meier curve and the associated patient stratification chart are illustrated at FIG. 7B.

TABLE 12
MODEL 3

| | | | | mfIHC |
|---|---|---|---|---|
| Feature Metric | ROI | HR | P-value | panel |
| MdD CD8+ from | TR | 2.103 | 0.0177 | 1 |
| PanCK+ w/in 30 μm | | | | |
| MnD CD8+ from | SR | 0.2258 | 0.0481 | 1 |
| PDL1+/PanCK+ w/in 10 μm | | | | |
| Mean # CD8+/PD1+ w/in | TR | 1.353e⁻¹³ | 0.0476 | 2 |
| 10 μm PDL1+/PanCK+ | | | | |
| MnD CD8+/PD1+ from | TR | 1.163 | 0.4903 | 2 |
| CD8+/PDL1+ w/in 30 μm | | | | |

| | |
|---|---|
| Model Evaluation | Likelihood ratio test = |
| | 27.27 on 7 df, p = 2e⁻⁰⁵ |

HR: hazard ratio;
MdD: median distance;
MnD: mean distance;
SDD: standard deviation of distance;
SR: stroma region;
TR: tumor region

### Example 3G: Model 4

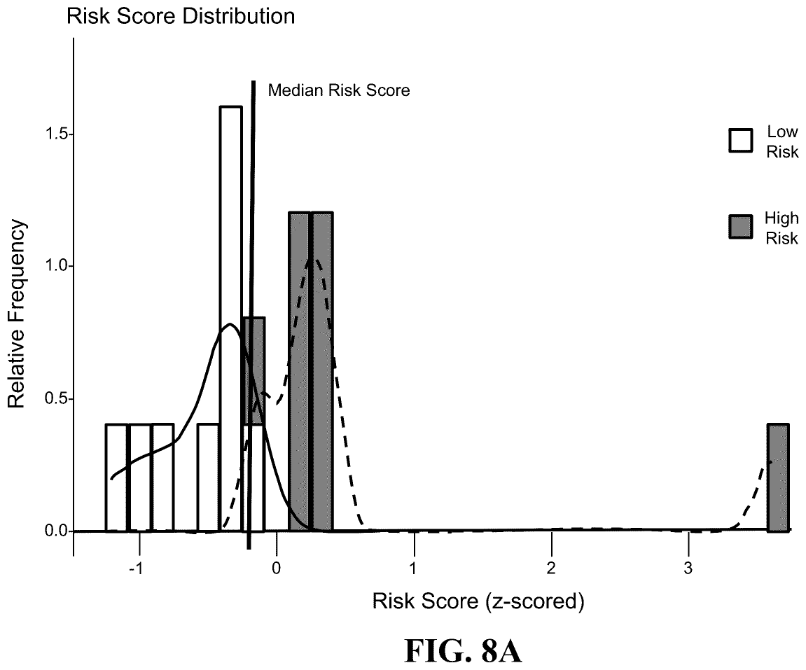

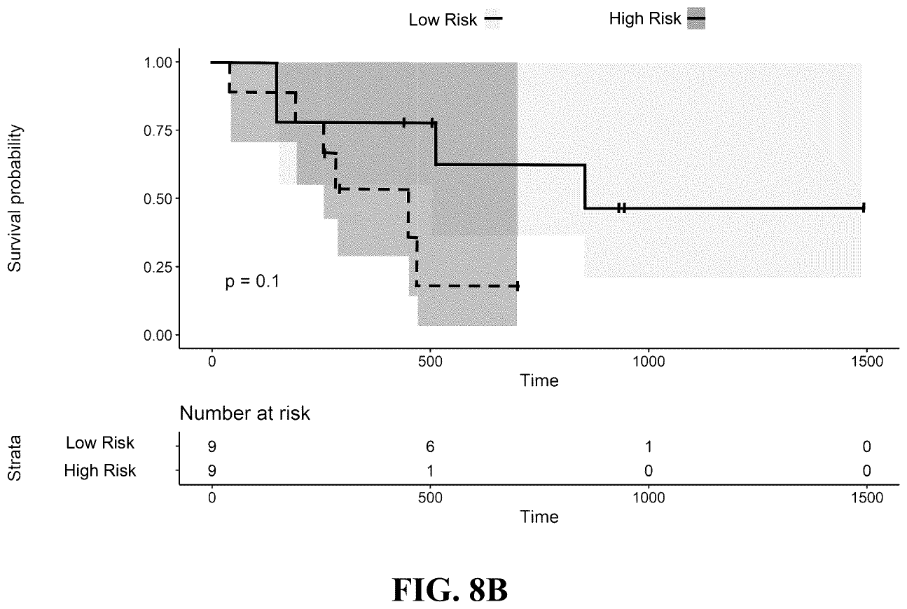

Model 4 was fit using a feature metric from panel A from Table 9 using progression-free survival as the relevant outcome. Model 4 details are summarized below at Table 13. The risk score distribution is illustrated at FIG. 8A (vertical black line illustrates stratification cutoff). A Kaplan-Meier curve and the associated patient stratification chart are illustrated at FIG. 8B.

TABLE 13
MODEL 4

| | | | | mfIHC |
|---|---|---|---|---|
| Feature Metric | ROI | HR | P-value | panel |
| Density of CD8+/PanCK− | TR | 0.1703 | 0.0408 | 2 |
| cells (100 cells/mm²) | | | | |

| | |
|---|---|
| Model Evaluation | Likelihood ratio test = |
| | 10.82 on 1 df, p = 0.001 |

HR: hazard ratio;
MdD: median distance;
MnD: mean distance;
SDD: standard deviation of distance;
SR: stroma region;
TR: tumor region

### Example 3H: Model 5

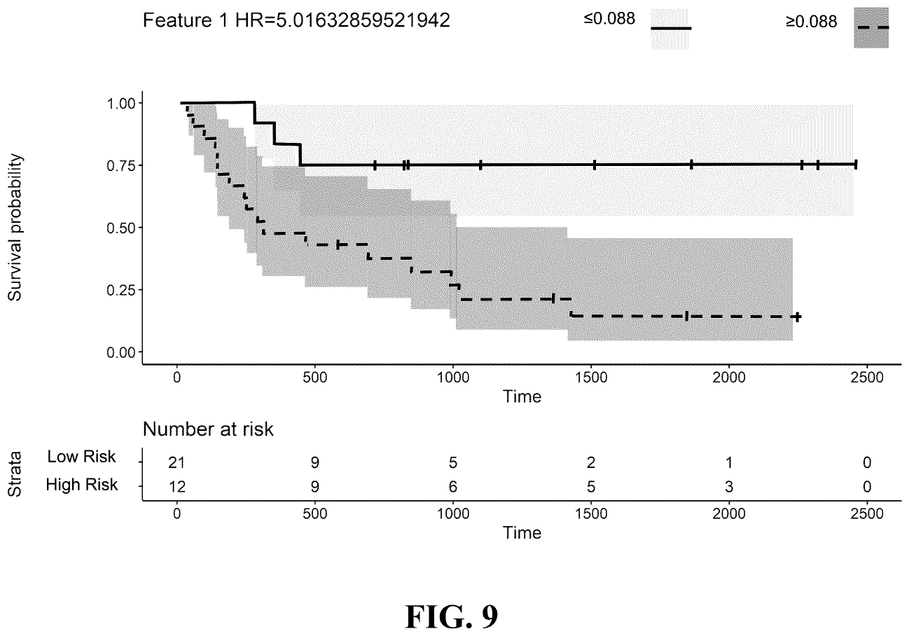

Model 5 was fit using the average number of PD-1+ cells within a pre-determined distance of at least 1 PD-L1+ cell from sample stained with panel B from Table 9 using progression-free survival as the relevant outcome. Multiple pre-determined distances were tested, including 5, 10, 15, 20, 25, 30, 35, 40, 45, and 50 μm. A radius of 10 μm had the highest significance. Model 5 details are summarized below at Table 14. The Kaplan-Meier curve and associated patient stratification for a feature values of 5.01 chart are illustrated in FIG. 9.

TABLE 14
MODEL 5

| | | | | mfIHC |
|---|---|---|---|---|
| Feature Metric | ROI | HR | P-value | panel |
| Average number of PD1+ cells | TR | 0.87 | 0.0408 | 2 |
| within 10 microns of a PD-L1+ | | | | |
| cell, within the Tumor region | | | | |

| | |
|---|---|
| Model Evaluation | Likelihood ratio test = |
| | 14.17 on 1 df, p = 0.00363 |

HR: hazard ratio;
TR: tumor region

### Example 4A: Validation Studies (Prophetic)

To confirm the predictive features discovered in Example 3, patient cases from an independent clinical series are studied. FFPE tumor tissue specimens from patients with MSI-H/dMMR mCRC treated with pembrolizumab are stained with the multiplex fluorescence marker panels as used in Example 3. Then the feature metrics for each of Models 1-5 are extracted from digitized slide images, and a risk score is computed for each patient case by combining these features.

Multiple cutoffs are evaluated for their ability to balance sensitivity and specificity of the models. Using the cutoff values, each patient case is assigned to either a low-risk group or a high-risk group. Survival outcomes in these subgroups are compared in order to confirm whether the models are indeed predictive in relation to the survival endpoints.

### Example 4B: Additional Validation Studies (Prophetic)

To confirm the predictive features of Model 5, a patient case from an independent clinical series are studied. FFPE tumor tissue specimens from patients with MSI-H/dMMR mCRC treated with a PD-1 axis-directed therapy are stained with a chromogenic duplex IHC assay for PD-1 and PD-L1. Then, the feature metrics for Model 5 are extracted from digitized slide images, and the average number of the average number of PD-1+ cells within a pre-determined distance of at least 1 PD-L1+ cell is computed for each patient case. Multiple pre-determined distances are tested for each patient sample.

Multiple cutoffs are evaluated for their ability to balance sensitivity and specificity of the models. Using the cutoff values, each patient case is assigned to either a low-risk group or a high-risk group. Survival outcomes in these subgroups are compared in order to confirm whether the models are indeed predictive in relation to the survival endpoints.

### REFERENCES

Aeffner et al., Introduction to Digital Image Analysis in Whole-slide Imaging: A White Paper from the Digital Pathology Association, Journal of Pathology Informatics, 2019, Vol. 10, No. 9, doi: 10.4103/jpi.jpi_82_18
Baharlou et al., Mass Cytometry Imaging for the Study of Human Diseases—Applications and Data Analysis Strategies, Frontiers in Immunology, 2019, Vol. 10, Art. 2657.
Barrera et al., Computer-extracted features relating to spatial arrangement of tumor infiltrating lymphocytes to predict response to nivolumab in non-small cell lung cancer (NSCLC). ASCO Annual Meeting 2018: Abstract #: 12115.
Bodenmiller, Multiplexed Epitope-Based Tissue Imaging for Discovery and Healthcare Applications, Cell Systems, 2016, Vol. 2, Issue 4, pp. 225-38.
Chakrabarti et al., Intratumoral CD3+ and CD8+ T-Cell Densities in Patients With DNA Mismatch Repair—Deficient Metastatic Colorectal Cancer Receiving Programmed Cell Death-1 Blockade, JCO Precision Oncology, 2019, Vol. 3, 1-7.
Galon et al., Validation of the Immunoscore (IM) as a prognostic marker in stage I/II/III colon cancer: Results of a worldwide consortium-based analysis of 1,336 patients, J. Clin. Oncol., Vol. 34, suppl. Abstract No. 3500 (2016) (“Galon I”).
Galon et al., Validation of the Immunoscore (IM) as a prognostic marker in stage I/II/III colon cancer: Results of a worldwide consortium-based analysis of 1,336 patients, 2016 ASCO Annual Meeting, Oral Abstract Session, Abstract #3500, (available at meetinglibrary.asco.org) (“Galon II”).
Gorris et al., Eight-Color Multiplex Immunohistochemistry for Simultaneous Detection of Multiple Immune Checkpoint Molecules within the Tumor Microenvironment, 2018, Journal of Immunology, Vol. 200, Issue 1, pp. 347-54.
Hofman et al., Multiplexed Immunohistochemistry for Molecular and Immune Profiling in Lung Cancer—Just About Ready for Prime-Time?, Cancers, 2019, Vol. 11, No. 283.
Ide et al., Chromogenic Multiplex Immunohistochemistry Reveals Modulation of the Immune Microenvironment Associated with Survival in Elderly Patients with Lung Adenocarcinoma, Cancers (Basel), 2018, Vol. 10, Issue 9, No. 326.
Jass, Lymphocytic infiltration and survival in rectal cancer, J. Clin. Pathol., 1986, Vol. 39, Issue 6, pp. 585-589 (Jass I).
Jass et al., A new prognostic classification of rectal cancer, The Lancet, 1987, Vol. 329, Issue 8545, pp. 1303-1306 (Jass II).
Le et al., PD-1 Blockade in Tumors with Mismatch-Repair Deficiency, New England Journal of Medicine, 2015, Vol. 372, Issue 26, pp. 2509-20 (Le I).
Le et al., Mismatch-repair deficiency predicts response of solid tumors to PD-1 blockade, Science, 10.1126/science.aan6733 (2017) (Le II).
Levenson, Immunohistochemistry and mass spectrometry for highly multiplexed cellular molecular imaging, Laboratory Investigation, 2015, Vol. 95, pp. 397-405.
Llosa et al., The vigorous immune microenvironment of microsatellite instable colon cancer is balanced by multiple counter-inhibitory checkpoints. Cancer discovery, 2015, Vol. 5, Issue 1, pp. 43-51.
Morrison et al., Brightfield multiplex immunohistochemistry with multispectral imaging, Laboratory Investigation, 2020, Vol. 100, pp. 1124-36.
Pagés et al., Immune infiltration in human tumors: a prognostic factor that should not be ignored, Oncogene, Vol. 29, pp. 1093-1102 (2010).
Parra et al., State-of-the-Art of Profiling Immune Contexture in the Era of Multiplexed Staining and Digital Analysis to Study Paraffin Tumor Tissues, Cancers, 2019, Vol. 11, Issue 2, No. 247.
Ptacek et al., Multiplexed ion beam imaging (MIBI) for characterization of the tumor microenvironment across tumor types, Laboratory Investigation, 2020, Vol. 100, pp. 1111-1123.
Stack et al., Multiplexed immunohistochemistry, imaging, and quantitation: A review, with an assessment of Tyramide signal amplification, multispectral imaging and multiplex analysis, 2014, Vol. 70, Issue 1, pp. 46-58.
Wang et al., Case classification with tumor antigen presenting and TGF-β signaling biomarkers to predict anti-PD-1 outcome in GI tract tumors using automated quantitative fluorescence multiplex IHC [abstract], Cancer Research, 2019, Vol. 79, Issue 13 (Suppl), Abstract 4030 (Wang I).
Wang et al., Exploration of PD-1/PD-L1 Spatial Interaction and T Cells Functionality to Predict Anti-PD1 Treatment Outcome in GI Tract Tumors using Automated Quantitative Fluorescence Multiplexed IHC, Society for Immunotherapy of Cancer Annual Meeting 2018, Poster #P703 (Wang II).
Wang, et al., Prediction of recurrence in early stage non-small cell lung cancer using computer extracted nuclear features from digital H&E images, Scientific Reports 7.1 (2017): 13543
WO 2020/072348 A1, Methods and systems for predicting response to pd-1 axis directed therapeutics, published 9 Apr. 2020.
WO 2020/161125 A1, Methods and systems for evaluation of immune cell infiltrate in stage iv colorectal cancer, published 13 Aug. 2020.
Yoon et al., Inter-tumoral heterogeneity of CD3 (+) and CD8 (+) T-cell densities in the microenvironment of DNA mismatch repair-deficient colon cancers: implications for prognosis. Clin Cancer Res., 2019, Vol. 25, Issue 1, pp. 125-133.
Zhang et al., Characterization of PD-L1, CD8, CD3, CD68 and PanCK in Tumor Microenvironment of GI Tract Tumors with respect to Patients' Mismatch Repair Status and Anti-PD-1 Treatment Outcome using 5Plex IHC and Whole Slide Image Analysis, Annals of Oncology, 2018, Vol. 29, Suppl. 8), VIII36-VIII37 (Zhang I).
Zhang et al., Fully automated 5-plex fluorescent immunohistochemistry with tyramide signal amplification and same species antibodies, Laboratory Investigation, 2017, Vol. 97, Issue 7, pp. 873-85 (Zhang II).
Zou et al., PD-L1 (B7-H1) and PD-1 pathway blockade for cancer therapy: Mechanisms, response biomarkers, and combinations. Science Translational Medicine, 2016, Vol. 8, Issue 328, 328rv324.

1. A method of treating a subject having a stage III or stage IV dMMR and/or MSI-H colorectal tumor, the method comprising administering to the subject a PD-1 axis-directed therapy, wherein the tumor has previously been determined to have an average number of PD-1⁺ cells within a pre-determined distance of a PD-L1⁺ cell that exceeds a pre-determined cutoff.

2. A method of selecting a subject having a stage III or stage IV dMMR and/or MSI-H colorectal tumor to receive a PD-1 axis directed therapeutic agent, the method comprising:
determining or having determined in a portion of the tumor an average number of PD-1⁺ cells within a pre-determined distance of a PD-L1⁺ cell;
comparing the average number of PD-1⁺ cells within the pre-determined distance of a PD-L1⁺ cell to a pre-determined cutoff;
selecting the subject to receive a PD-1 axis directed therapeutic agent if the average number of PD-1⁺ cells within a pre-determined distance of a PD-L1⁺ cell exceeds the pre-determined cutoff.

3. The method of claim 1, wherein the pre-determined distance is in the range selected from the group consisting of: 5 μm to 50 μm, 5 μm to 40 μm, 5 μm to 30 μm, 5 μm to 25 μm, 5 μm to 20 μm, 5 μm to 15 μm, 5 μm to 10 μm, 10 μm to 50 μm, 10 μm to 40 μm, 10 μm to 30 μm, 10 μm to 25 μm, 10 μm to 20 μm, and 10 μm to 15 μm.

4.-5. (canceled)

6. The method of claim 1, wherein the average number of PD-1⁺ cells within the pre-determined distance of a PD-L1⁺ cell is determined in a tissue section of the tumor, wherein the tumor is affinity histochemically stained for each of human PD-1 and human PD-L1.

7.-10. (canceled)

11. A method of treating a subject having a dMMR and/or MSI-H colorectal tumor, the method comprising administering to the subject a PD-1 axis-directed therapy,
(a) wherein the tumor has previously been determined to have an Predicted Response Score (PRS) indicative of response to the PD-1 axis-directed therapy and wherein the PRS is determined from a continuous scoring function incorporating a feature set comprising at least an average number of PD-1⁺ cells within 10 μm of a PD-L1 cell within the tumor region of a tissue section of the tumor; or
(b) wherein the tumor has previously been determined to have a Predicted Response Score (PRS) indicative of response to the PD-1 axis directed therapy and wherein the PRS determined from a continuous scoring function incorporating a feature set comprising at least:
(i) standard deviation of the distance of CD8+ cells from CD68+/PDL1+ cells within 10 μm in a stroma region of a tissue section of the tumor, and
(ii) the mean distance of CD8+/PD-1+ cells from CD8+-PD-L1+ cells within 30 μm in a tumor region of a tissue section of the tumor; or
(c) wherein the tumor has previously been determined to have a Predicted Response Score (PRS) indicative of response to the PD-1 axis-directed therapy and wherein the PRS determined from a continuous scoring function incorporating a feature set comprising at least:
(i) standard deviation of the distance of CD8+ cells from CD68+/PDL1+ cells within 10 μm in a peritumor outside region of a tissue section of the tumor, and
(ii) density of CD8+/epithelial marker− (EM−) cells within a tumor area; or
(d) wherein the tumor has previously been determined to have a Predicted Response Score (PRS) indicative of response to the PD-1 axis-directed therapy and wherein the PRS determined from a continuous scoring function incorporating a feature set comprising at least:
(i) median distance of CD8+ cells from epithelial marker+ (EM+) cells within 30 μm in a epithelial tumor region of a tissue section of the tumor,
(ii) mean distance of CD8+ cells from PD-L1+/epithelial marker+ (EM+) cells within 10 μm a tumor stroma area,
(iii) mean number CD8+/PD-1+ cells from CD8+/PD-L1+ cells within 10 μm in a tumor area, and
(iv) mean distance of CD8+/PD-1+ cells from CD8+/PD-L1+ cells within 30 μm in a tumor area.

12. The method of claim 11, wherein the tissue section of (a) is affinity histochemically stained for each of PD-1 and PD-L1.

13.-23. (canceled)

24. The method of claim 11, wherein the tissue section is histochemically stained for one or more of CD8, PD-L1, PD-1, and an epithelial marker.

25. The method of claim 1, wherein the PD-1 axis-directed therapy comprises an anti-PD-1 antibody or an anti-PD-L1 antibody.

26.-29. (canceled)

30. An image analysis system comprising a memory and a processor, wherein the processor implements a set of instructions stored on the memory, the set of instructions comprising extracting a Feature Set from one or more digital images of an affinity histochemically (AHC) labeled tissue sample from a colorectal tumor previously determined to be one or more of dMMR or MSI-H, wherein the Feature Set is selected from the group consisting of:
(a) Feature Set 1, comprising:
a standard deviation of the distance of CD8+ cells from CD68+/PDL1+ cells within 10 μm in a stroma region, and
a mean distance of CD8+/PD-1+ cells from CD8+-PD-L1+ cells within 30 μm in a tumor region;
(b) Feature Set 2, comprising:
a standard deviation of the distance of CD8+ cells from CD68+/PDL1+ cells within 10 μm in a peritumor outside region, and
a density of CD8+/epithelial marker− (EM−) cells within a tumor area;
(c) Feature Set 3, comprising:
a median distance of CD8+ cells from epithelial marker+ (EM+) cells within 30 μm in a epithelial tumor region of a tissue section of the tumor,
a mean distance of CD8+ cells from PD-L1+/epithelial marker+ (EM+) cells within 10 μm a stroma area,
a mean number CD8+/PD-1+ cells from CD8+/PD-L1+ cells within 10 μm in a tumor area, and
a mean distance of CD8+/PD-1+ cells from CD8+/PD-L1+ cells within 30 μm in a tumor area, and
(d) Feature Set 4, comprising a density of CD8+ cells in a tumor area; and
(e) Feature Set 5, comprising an average number of PD-1⁺ cells within 10 μm of a PD-L1⁺ cell.

31. The image analysis system of claim 30, wherein the Feature Set is extracted by implementing a Region of Interest (ROI) Module, a Feature Identification (FI) Module, and a Scoring Module on the digital image(s), wherein the ROI module annotates one or more ROI on the digital image(s), the FI Module marks cells at least in the relevant ROIs and generates a feature vector for each cell with a status of a relevant biomarker, and the Scoring Module extracts relevant the feature metrics from the relevant ROIs.

32. The image analysis system of claim 31, wherein the image analysis system extracts the feature metrics of Feature Set 1 from a digital image of an mAHC-labeled tissue sample, wherein:
(a1) the ROI Module annotates a stroma region and a tumor region on the digital image;
(a2) the FI Module:
(a2a) marks objects corresponding to cells in the digital image;
(a2b) generates a feature vector indicating CD8, CD68, PD-L1, and optionally EM status for at least the cells in the stroma region; and
(a2c) generates a feature vector indicating CD8, PD-1, PD-L1, and optionally EM status for at least the cells in the tumor region; and
(a3) the Scoring Module extracts the feature metrics of Feature Set 1 from their respective ROIs.

33.-34. (canceled)

35. The image analysis system of claim 31, wherein the image analysis system extracts the feature metrics of Feature Set 1 from digital images from a first digital image of a first AHC-labeled section of a tumor and a second digital image of a second AHC-labeled section of the tumor, wherein:
(a1) a first ROI Module annotates a stroma region in the first digital image;
(a2) a first FI Module marks objects corresponding to cells at least in the stroma region and generates a feature vector for each marked cell indicating CD8, CD68, PD-L1, and optionally EM status;
(a3) a first Scoring Module 206 computes a standard deviation of distance of CD8+ cells from CD68+/PDL1+ cells within 10 μm in the stroma region of the first digital image;
(a4) a second ROI Module 205 annotates a tumor region on the second digital image;
(a5) a second FI Module 204 marks objects corresponding to cells at least in the tumor region of the second digital image and generates a feature vector indicating CD8, PD-1, PD-L1, and optionally EM status for at least the cells in the tumor region;
(a6) a second Scoring Module 206 computes a mean distance of CD8+/PD-1+ cells from CD8+/PD-L1+ cells w/in 30 μm in the tumor region of the second digital image.

36.-37. (canceled)

38. The image analysis system of claim 31, wherein the image analysis system extracts the feature metrics of Feature Set 2 from a digital image of an mAHC-labeled tissue sample, wherein:
(b1) the ROI Module annotates a peritumor outside (PO) region and a tumor region on the digital image;
(b2) the FI Module:
(b2a) marks objects corresponding to cells in the digital image;
(b2b) generates a feature vector indicating CD8, CD68, PD-L1, and optionally EM status for at least the cells in the PO region; and
(b2c) generates a feature vector indicating CD8 and optionally EM status for at least the cells in the tumor region; and
(c) the Scoring Module extracts the feature metrics of Feature Set 2 from their respective ROIs; or wherein
the image analysis system extracts the feature metrics of Feature Set 2 from a first digital image of a first AHC-labeled section of a tumor and a second digital image of a second AHC-labeled section of the tumor, wherein:
(b1) a first ROI Module annotates a peritumor outer (PO) region in the first digital image;
(b2) a first FI Module marks objects corresponding to cells at least in the PO region and generates a feature vector for each marked cell indicating CD8, CD68, PD-L1, and optionally EM status;
(b3) a first Scoring Module extracts a standard deviation of distance of CD8+ cells from CD68+/PDL1+ cells within 10 μm in the PO region of the first digital image;
(b4) a second ROI Module annotates a tumor region on the second digital image;
(b5) a second FI Module marks objects corresponding to cells at least in the tumor region of the second digital image and generates a feature vector indicating CD8 and optionally EM status for at least the cells in the tumor region; and
(b6) a second Scoring Module computes a density of CD8+ cells in the tumor region of the second digital image.

39.-42. (canceled)

43. The image analysis system of claim 31, wherein the image analysis system extracts the feature metrics of Feature Set 3 from a digital image of an mAHC-labeled tissue sample, wherein:
(c1) the ROI Module annotates a tumor region and a stroma region on the digital image;
(c2) the FI Module
(c2a) marks objects corresponding to cells in the digital image,
(c2b) generates a feature vector indicating CD8, PD-1, PD-L1, and EM status for at least the cells in the tumor region, and
(c2c) generates a feature vector indicating CD8, PD-L1, and EM status for at least the cells in the stroma region; and
(c3) the Scoring Module extracts the feature metrics of Feature Set 3 from their respective ROIs.

44.-45. (canceled)

46. The image analysis system of claim 31, wherein the image analysis system extracts the feature metrics of Feature Set 3 from a first digital image of a first AHC-labeled section of a tumor and a second digital image of a second AHC-labeled section of the tumor, wherein:
(b1) a first ROI Module annotates a tumor region in the first digital image;
(b2) a first FI Module marks objects corresponding to cells at least in the tumor region and generates a feature vector for each marked cell indicating CD8, PD-1, PD-L1, and EM status;
(b3) a first Scoring Module computes (c1) a median distance from CD8+ cells to EM+ cells within 30 μm, (c2) a mean number of CD8+/PD-1+ cells within 10 μm of CD8+/PD-L1+ cells, and (c3) a mean distance from CD8+/PD-1+ cells to CD8+/PD-L1+ cells within 30 μm from the tumor region of the first digital image;
(b4) a second ROI Module annotates a stroma region on the second digital image;
(b5) a second FI Module marks objects corresponding to cells at least in the stroma region of the second digital image and generates a feature vector indicating CD8, PD-L1, and EM status for at least the cells in the stroma region; and
(b6) a second Scoring Module computes mean distance from CD8+ cells to PD-L1+/EM+ cells within 10 μm in the stroma region in the tumor region of the second digital image.

47.-48. (canceled)

49. The image analysis system of claim 31, wherein the image analysis system extracts the feature metrics of Feature Set 3 from a first digital image of a first AHC-labeled section of a tumor and a second digital image of a second AHC-labeled section of the tumor, wherein:
(c1) a first ROI Module annotates a tumor region and a stroma region in the first digital image;
(c2) a first FI Module:
(c2a) marks objects corresponding to cells in the digital image,
(c2b) generates a feature vector for at least each marked cell in the tumor region indicating CD8 and EM status, and
(c2c) generates a feature vector for at least each marked cell in the stromal region indicating CD8, PD-L1, and EM status;
(c3) a first Scoring Module computes (c1) median distance of CD8+ cells from EM+ cells w/in 30 μm in the tumor region of the first digital image and (c2) median distance from CD8+ cells to PD-L1+/epithelial marker+ (EM+) cells within 10 μm in the stroma region of the first digital image;
(c4) a second ROI Module annotates a tumor region on the second digital image;
(c5) a second FI Module marks objects corresponding to cells at least in the tumor region of the second digital image and generates a feature vector indicating CD8, PD-1, PD-L1, and optionally EM status for at least the cells in the tumor region;
(c6) a second Scoring Module computes
(c6a) a mean number of CD8+/PD-1+ cells within 10 μm of CD8+/PD-L1+ cells in the tumor region of the second digital image and
(c6b) a distance from CD8+/PD-1+ cells to CD8+/PD-L1+ cells within 30 μm in the tumor region of the second digital image.

50.-51. (canceled)

52. The image analysis system of claim 31, wherein the image analysis system extracts the feature metrics of Feature Set 3 from a first digital image of a first AHC-labeled section of a tumor, a second digital image of a second AHC-labeled section of the tumor, and a third digital image of a third AHC-labeled section of a tumor wherein:
(c1) a first ROI Module annotates a tumor region in the first digital image;
(c2) a first FI Module marks objects corresponding to cells in at least the tumor region of the first digital image and generates a feature vector for at least each marked cell in the tumor region indicating CD8 and EM status;
(c3) a first Scoring Module computes a median distance of CD8+ cells from EM+ cells w/in 30 μm in the tumor region of the first digital image;
(c4) a second ROI Module annotates a stroma region on the second digital image;
(c5) a second FI Module marks objects corresponding to cells at least in the stroma region of the second digital image and generates a feature vector indicating CD8, PD-L1, and EM status for at least the cells in the stroma region;
(c6) a second Scoring Module computes median distance from CD8+ cells to PD-L1+/EM+ cells within 10 μm in the stroma region of the second digital image;
(c7) a third ROI Module annotates a tumor region on the third digital image;
(c8) a third FI Module marks objects corresponding to cells at least in the tumor region of the third digital image and generates a feature vector indicating CD8, PD-1, PD-L1, and optionally EM status for at least the cells in the tumor region; and
(c9) a third Scoring Module computes
(c9a) a mean number of CD8+/PD-1+ cells within 10 μm of CD8+/PD-L1+ cells and
(c9b) a median distance from CD8+/PD-1+ cells to CD8+/PD-L1+ cells within 30 μm.

53.-54. (canceled)

55. The image analysis system of claim 31, wherein the image analysis system extracts the feature metrics of Feature Set 4 from a digital image of an AHC-labeled tissue sample, wherein:
(d1) the ROI Module annotates a tumor region on the digital image;
(d2) the FI Module marks objects corresponding to cells in the digital image and generates a feature vector indicating CD8 and optionally EM status for at least the cells in the tumor region; and
(d3) the Scoring Module extracts the feature metrics of Feature Set 4 from their respective ROI.

56.-58. (canceled)

59. The image analysis system of claim 31, wherein the image analysis system extracts the feature metrics of Feature Set 5 from a digital image of an AHC-labeled tissue sample, wherein:
(d1) the ROI Module annotates a tumor region on the digital image;
(d2) the FI Module marks objects corresponding to cells in the digital image and generates a feature vector indicating PD-1 and PD-L1 status for at least the cells in the tumor region; and
(d3) the Scoring Module extracts the feature metrics of Feature Set 5 from their respective ROI.

60.-69. (canceled)

70. A biomarker-specific reagent panel for predicting response to a PD-1 axis-directed therapy, the panel selected from the group consisting:
Panel A, comprising biomarker specific reagents specific for CD8, CD68, PD-L1, PD-1, and optionally epithelial marker (EM) proteins;
Panel B, comprising biomarker specific reagents specific for CD8, CD68, PD-L1, and optionally EM proteins;
Panel C, comprising biomarker specific reagents specific for CD8, EM, PD-L1, and PD-1 proteins;
Panel D, comprising biomarker specific reagents specific for CD8 and EM proteins;
Panel E, comprising biomarker specific reagents specific for CD8, PD-L1, PD-1, and optionally EM proteins;
Panel F, comprising biomarker specific reagents specific for CD8, PD-L1, and EM proteins; and
Panel G, comprising biomarker specific reagents specific for PD-1 and PD-L1.

71. The biomarker-specific reagent panel of claim 70, wherein the EM is pan-cytokeratin, wherein the biomarker specific reagents comprise antibodies or antibody fragments, and/or wherein the panels are suitable for a multiplex AHC assay including each of the recited markers.

72.-73. (canceled)

74. A multiplex affinity histochemical (mAHC) assay-stained tissue section of a colorectal tumor previously determined to be one or more of dMMR or MSI-H, wherein the tissue section is differentially stained for a panel of biomarkers selected from the group consisting of:
Panel A, wherein the AHC-stained tissue section is differentially stained for each of CD8, CD68, PD-L1, PD-1, and optionally epithelial marker (EM) proteins;
Panel B, wherein the AHC-stained tissue section is differentially stained for each of CD8, CD68, PD-L1, and optionally EM proteins;
Panel C, wherein the AHC-stained tissue section is differentially stained for each of CD8, EM, PD-L1, and PD-1 proteins;
Panel D, wherein the AHC-stained tissue section is differentially stained for each of CD8 and EM proteins;
Panel E, wherein the AHC-stained tissue section is differentially stained for each of CD8, PD-L1, PD-1, and optionally EM proteins;
Panel F, wherein the AHC-stained tissue section is differentially stained for each of CD8, PD-L1, and EM proteins; and
Panel G, wherein the AHC-stained tissue section is differentially stained for each of PD-1 and PD-L1 proteins.

75. The mAHC-stained tissue section of claim 74, wherein the mAHC assay is a multiplex immunohistochemical assay (mIHC).

76.-77. (canceled)

78. The method of claim 2, wherein the method further comprises treating the subject having an average number of PD-1⁺ cells within a pre-determined distance of a PD-L1⁺ cell exceeding the pre-determined cutoff with a PD-1 axis directed therapeutic agent.

79. The method of claim 6, wherein:
i) human PD-1 is affinity histochemically stained with a first brightfield dye and human PD-L1 is affinity histochemically stained with a second brightfield dye,
ii) the tissue section is a formalin-fixed, paraffin embedded tissue section,
iii) the average number of PD-1⁺ cells within the pre-determined distance of the PD-L1⁺ cell is extracted from a digital image of the affinity histochemically stained tissue section by an image analysis system, and/or
iv) the tumor is affinity histochemically stained with an anti-human PD-1 monoclonal antibody and an anti-human PD-L1 monoclonal antibody.
